SEQUENCE LISTING (86 sequences)

SEQ ID NO: 1 - DNA, 3903 bp, synthetic construct
  source 1..3903: mol_type = other DNA
atgtcaatttatcaagaatttgtgaacaaatatagcctgagcaaaaccctgcgttttgaactgattccgcagggtaaaaccctggaaaacattaaagcacgtggtctgattctggatgatgaaaaacgtgccaaagactacaaaaaagccaaacaaatcatcgataaataccaccagttcttcatcgaagaaattctgagcagcgtttgcattagcgaagatctgctgcagaattattccgacgtttatttcaaactgaaaaaaagcgacgatgataacctgcagaaagatttcaaaagcgccaaagataccatcaaaaaacaaattagcgagtatatcaaagacagcgagaaattcaaaaacctgttcaaccagaatctgatcgatgccaaaaaaggtcaagaaagcgatctgatcctgtggctgaaacagagcaaagataatggcatcgaactgtttaaagccaacagcgatattaccgatattgatgaagcactggaaatcatcaaaagctttaaaggttggaccacctactttaaaggctttcacgaaaatcgcaaaaacgtgtatagcagcaatgatattccgaccagcattatctatcgcatcgttgatgataatctgcctaaatttctggaaaataaagccaaatatgaaagcctgaaagacaaagcaccggaagcaattaactatgagcagatcaaaaaagatctggccgaagaactgacctttgacattgattacaaaaccagcgaagttaaccagcgtgtttttagcctggatgaagtttttgaaattgccaacttcaacaactacctgaatcagagcggtatcaccaaattcaataccattatcggtggcaaattcgtgaatggcgaaaataccaaacgcaaaggcatcaacgaatacattaatctgtatagccagcagattaacgataaaacgctgaaaaaatacaaaatgagcgtgctgttcaaacaaattctgtcagataccgaaagcaaaagcttcgtgattgacaaactggaagatgatagtgatgttgttaccaccatgcagagcttttatgaacaaatcgcagcgtttaaaaccgtggaagagaaatccattaaagaaaccctgagcctgctgtttgatgatctgaaagcacagaaactggacctgtccaaaatctacttcaaaaacgataaatccctgaccgatctgagccagcaggttttcgatgattatagcgttattggcaccgcagttctggaatatatcacacagcagattgcaccgaaaaatctggataatccgagcaaaaaagaacaagagctgatcgccaaaaaaaccgagaaagcgaaatatctgagcctggaaacaattaaactggccctggaagaatttaacaaacaccgcgacattgataaacagtgccgctttgaagaaatcctggcaaattttgcagcaatcccgatgatctttgatgaaattgcgcagaataaagataacctggcacagatcagcatcaaatatcagaatcagggaaaaaaagacctgctgcaagcaagtgccgaagatgatgttaaagcgattaaagatctgctggatcagaccaataacctgctgcataaactgaaaatctttcacattagccagagcgaggataaagcgaacattctggataaagatgagcacttctatctggtgtttgaagagtgttattttgagctggcaaatattgtgccgctgtataacaaaatccgcaactatattacccagaaaccgtatagcgacgaaaaattcaaactgaactttgagaatagcaccctggccaatggttgggataaaaacaaagaaccggataataccgccatcctgttcattaaagatgataaatactatctgggcgtgatgaacaaaaaaaacaacaaaatcttcgacgataaagccatcaaagagaataaaggcgaaggttacaaaaaaatcgtgtacaaactgctgcctggtgcgaataaaatgctgccgaaagtgttttttagcgccaaatccatcaaattctataacccgagcgaagatattctgcgtattcgtaatcatagcacccataccaaaaatggtagtccgcagaaaggctatgaaaaattcgagttcaacattgaggattgccgcaaattcatcgacttctacaaacagtccattagcaaacatccggaatggaaagactttggttttcgttttagcgatacccagcgctataacagcattgatgaattttatcgcgaagtggaaaaccagggctataaactgacatttgaaaacatcagcgagagctatattgatagcgttgtgaatcagggtaaactgtacctgtttcagatctataacaaagactttagcgcctatagcaaaggtcgtccgaatctgcataccctgtattggaaagcactgttcgatgaacgtaatctgcaggatgttgtctacaaactgaatggtgaagcagaactgttttatcgcaaacagagtatcccgaaaaaaatcacccatccggcaaaagaagcaatcgcgaacaaaaacaaagataacccgaaaaaagaaagcgtgttcgagtatgatctgatcaaagataaacgcttcaccgaagataaattctttttccattgcccgatcaccatcaactttaaaagcagcggtgcgaacaaattcaacgatgaaatcaatctgctgctgaaagaaaaagccaacgatgttcatattctgagcattgatcgtggtgaacgtcatctggcctattacaccctggttgatggtaaaggcaatattatcaaacaggacaccttcaacattatcggcaatgatcgtatgaaaaccaactaccatgataaactggcagccattgaaaaagatcgtgatagcgcacgtaaagattggaaaaaaatcaacaacattaaagaaatgaaagaaggctacctgagccaggttgttcatgaaatcgccaaactggtgattgaatataatgccattgtggtgttcgaggatctgaacttcggtttcaaacgtggtcgtttcaaagttgagaaacaggtgtatcaaaaactggaaaaaatgctgatcgaaaaactgaattacctggtgttcaaagacaacgaattcgataaaaccggtggtgttctgcgtgcatatcagctgaccgcaccttttgaaaccttcaaaaaaatgggtaaacagaccggcatcatctattatgttccggcaggttttacctccaaaatttgtccggttaccggctttgttaatcagctgtatccgaaatatgagagcgttagcaaaagccaagagtttttcagcaaatttgataaaatctgctataacctggacaaaggctactttgaattcagctttgactataaaaactttggcgataaagcagccaaaggcaaatggaccattgcaagctttggtagccgtctgattaactttcgtaacagcgacaaaaaccataactgggatacccgtgaagtttatccgaccaaagagctggaaaaactgctgaaagattacagcattgaatatggtcatggcgaatgtattaaagccgcaatttgtggtgagtccgacaaaaaattctttgcaaaactgaccagcgtgctgaataccattctgcagatgcgtaatagcaaaaccggcaccgaactggattatctgattagtccggttgcagatgtgaacggcaattttttcgatagccgtcaggctccgaaaaatatgccgcaggatgcagatgcaaatggtgcctatcatattggcctgaaaggtctgatgctgctgggtcgcattaaaaacaatcaagaaggcaaaaaactgaacctggtgatcaaaaacgaagagtattttgagttcgtgcagaataggaataactaa

SEQ ID NO: 2 - DNA, 3403 bp, synthetic construct
  source 1..3403: mol_type = other DNA
atgagcacaaaaccactcttcctgttacggaaagcgaaaaaatcatccggtgaacctgacgtcgtcctgtgggcaagcaacgattttgaatcgacctgtgccactctggactacctgatcgttaagtcaggtaaaaaactgagcagctattttaaagctgttgccacgaattttcctgtcgttaatgacctgcccgctgaaggtgagatcgattttacctggagtgaacgctatcaactcagcaaagactccatgacatgggaactaaaaccgggagcagcaccagacaacgctcactatcaaggcaataccaacgtcaacggcgaagacatgactgagattgaggagaatatgctactcccaatttctggccaggaactgcccattcgttggcttgctcaacacggcagcgaaaaaccggtaacgcacgtttcacgcgacggactccaggcattacacattgctcgggctgaagaactaccggctgttactgccctggctgtttcccacaaaaccagcctgctcgacccgctggaaattcgcgaactccacaaactggttcgtgacactgacaaagttttccctaatcctggtaattcaaacctgggactgataactgcttttttcgaagcatacctgaacgctgactacaccgatcgaggactgctgacaaaagagtggatgaagggtaatcgtgtttcacacatcactcgcacggcttccggtgctaatgctggcggcggaaacctcaccgatcgcggcgaaggtttcgtacacgatctgacgtcactggcgcgcgacgtagccactggcgtactggcccgttcaatggatctggacatctataaccttcatccggcacacgctaaacgcattgaggaaattatcgctgaaaataaaccgcccttttctgttttccgcgacaaattcatcaccatgcctggcgggctggattattcccgcgccatcgtggttgcgtccgtaaaagaagcaccaattgggatcgaggtcatccccgcgcacgtcactgaatatctgaacaaagtactgactgaaaccgatcatgccaaccctgatccggaaatcgtggatattgcctgcggtcgctcctctgccccgatgccgcagcgagtaacagaagaaggaaaacaggatgatgaagaaaaaccgcaaccatctggaacaacggcagttgaacagggagaggctgaaacaatggaaccggacgcaactgaacatcatcaggacacgcagccgctggatgctcagtcacaggtaaattctgttgatgcgaaatatcaggaactgcgggcagaactccatgaagcccggaaaaacattccatcaaaaaatcctgtcgatgacgataaattgcttgctgcatcacgtggtgaatttgttgacggaattagcgacccgaacgatccgaaatgggtaaaggggatccagactcgcgattgtgtgtaccagaaccagccagaaacggaaaaaaccagcccagatatgaatcaacctgagccagtagtgcaacaggaaccggaaatagcctgcaatgcctgcggccagactggcggggataactgccctgactgtggtgcggtgatgggcgacgcaacataccaggaaacattcgatgaagagagtcaggttgaagctaaggaaaatgatccggaggaaatggaaggcgctgaacatccgcacaatgagaatgctggcagcgatccgcatcgcgattgcagtgatgaaactggcgaagtcgcagatcccgtaatcgtagaagacatagagccaggtatttattacggaatttcgaatgagaattaccacgcgggtcccggtatcagtaagtctcagctcgatgacattgctgatactccggcactatatttgtggcgtaaaaatgcccccgtggacaccacaaagacaaaaacgctcgatttaggaactgctttccactgccgggtacttgaaccggaagaattcagtaaccgctttatcgtagcacctgaatttaaccgccgtacaaacgccggaaaagaagaagagaaagcgtttctgatggaatgcgcaagcacaggaaaaacggttatcactgcggaagaaggccggaaaattgaactcatgtatcaaagcgttatggctttgccgctggggcaatggcttgttgaaagcgccggacacgctgaatcatcaatttactgggaagatcctgaaacaggaattttgtgtcggtgccgtccggacaaaattatccctgaatttcactggatcatggacgtgaaaactacggcggatattcaacgattcaaaaccgcttattacgactaccgctatcacgttcaggatgcattctacagtgacggttatgaagcacagtttggagtgcagccaactttcgtttttctggttgccagcacaactattgaatgcggacgttatccggttgaaattttcatgatgggcgaagaagcaaaactggcaggtcaacaggaatatcaccgcaatctgcgaaccctgtctgactgcctgaataccgatgaatggccagctattaagacattatcactgccccgctgggctaaggaatatgcaaatgactaagcaaccaccaatcgcaaaagccgatctgcaaaaaactcagggaaaccgtgcaccagcagcagttaaaaatagcgacgtgattagttttattaaccagccatcaatgaaagagcaactggcagcagctcttccacgccatatgacggctgaacgtatgatccgtatcgccaccacagaaattcgtaaagttccggcgttaggaaactgtgacactatgagttttgtcagtgcgatcgtacagtgttcacagctcggacttgagccaggtagcgccctcggtcatgcatatttactgccttttggtaataaaaacgaaaagagcggtaaaaagaacgttcagctaatcattggctatcgcggcatgattgatctggctcgccgttctggtcaaatcgccagcctgtcagcccgtgttgtccgtgaaggtgacgagtttagcttcgaatttggccttgatgaaaagttaatacaccgcccgggagaaaacgaagatgccccggttacccacgtctatgctgtcgcaagactgaaagacggaggtactcagtttgaagttatgacgcgcaaacagattgagctggtgcgcagcctgagtaaagctggtaataacgggccgtgggtaactcactgggaagaaatggcaaagaaaacggctattcgtcgcctgttcaaatatttgcccgtatcaattgagatccagcgtgcagtatcaatggatgaaaaggaaccactgacaatcgatcctgcagattcctctgtattaaccggggaatacagtgtaatcgataattcagaggaataa

SEQ ID NO: 3 - DNA, 736 bp, synthetic construct
  source 1..736: mol_type = other DNA
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcgttgatagagttattttaccactccctatcagtgatagagaaaagtattcaaatgatctaaagaggagaaaggatctccc

SEQ ID NO: 4 - DNA, 1199 bp, synthetic construct
  source 1..1199: mol_type = other DNA
ttatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccataaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccataagtattcaaatgatctaaagaggagaaaggatctccc

SEQ ID NO: 5 - DNA, 55 bp, synthetic construct
  source 1..55: mol_type = other DNA
tttaagtaaaaggctcgatattattcctctgaattatcgattacactgtattccc

SEQ ID NO: 6 - DNA, 44 bp, synthetic construct
  source 1..44: mol_type = other DNA
aagaggagaaaggatctcccatgagcacaaaaccactcttcctg

SEQ ID NO: 7 - DNA, 23 bp, synthetic construct
  source 1..23: mol_type = other DNA
gggagatcctttctcctctttag

SEQ ID NO: 8 - DNA, 47 bp, synthetic construct
  source 1..47: mol_type = other DNA
agctgtcaaacatgagaattttaagacccactttcacatttaagttg

SEQ ID NO: 9 - DNA, 44 bp, synthetic construct
  source 1..44: mol_type = other DNA
agctgtcaaacatgagaattttatgacaacttgacggctacatc

SEQ ID NO: 10 - DNA, 40 bp, synthetic construct
  source 1..40: mol_type = other DNA
taagggattttggtcatgagcggatgctgctttggatttg

SEQ ID NO: 11 - DNA, 35 bp, synthetic construct
  source 1..35: mol_type = other DNA
tatcgagccttttacttaaaataatctatcgaaag

SEQ ID NO: 12 - DNA, 29 bp, synthetic construct
  source 1..29: mol_type = other DNA
gctttttgaaaaagccgtttcctgtattg

SEQ ID NO: 13 - DNA, 46 bp, synthetic construct
  source 1..46: mol_type = other DNA
tcacatgttctttcctgcgtttaccagcatgttttatcaggaatcg

SEQ ID NO: 14 - DNA, 28 bp, synthetic construct
  source 1..28: mol_type = other DNA
aattctcatgtttgacagcttatcatcg

SEQ ID NO: 15 - DNA, 41 bp, synthetic construct
  source 1..41: mol_type = other DNA
aaacggctttttcaaaaagcttacgccccgccctgccactc

SEQ ID NO: 16 - DNA, 22 bp, synthetic construct
  source 1..22: mol_type = other DNA
acgcaggaaagaacatgtgagc

SEQ ID NO: 17 - DNA, 25 bp, synthetic construct
  source 1..25: mol_type = other DNA
ctcatgaccaaaatcccttaacgtg

SEQ ID NO: 18 - DNA, 55 bp, synthetic construct
  source 1..55: mol_type = other DNA
aagaggagaaaggatctcccatgtcaatttatcaagaatttgtgaacaaatatag

SEQ ID NO: 19 - DNA, 43 bp, synthetic construct
  source 1..43: mol_type = other DNA
ccgtctggcgtcgggcgtgataaaacgaaaggcccagtctttc

SEQ ID NO: 20 - DNA, 44 bp, synthetic construct
  source 1..44: mol_type = other DNA
taagggattttggtcatgagttaggcgagaagggaaagggcaag

SEQ ID NO: 21 - DNA, 37 bp, synthetic construct
  source 1..37: mol_type = other DNA
tcgttaaatattcagatagacggagataataaacggg

SEQ ID NO: 22 - DNA, 18 bp, synthetic construct
  source 1..18: mol_type = other DNA
tcacgcccgacgccagac

SEQ ID NO: 23 - DNA, 44 bp, synthetic construct
  source 1..44: mol_type = other DNA
tcacatgttctttcctgcgtcaccctctggtgattgtcgatatc

SEQ ID NO: 24 - DNA, 48 bp, synthetic construct
  source 1..48: mol_type = other DNA
tctatctgaatatttaacgaaattctcatgtttgacagcttatcatcg

SEQ ID NO: 25 - DNA, 22 bp, synthetic construct
  source 1..22: mol_type = other DNA
ttatttgccgactaccttggtg

SEQ ID NO: 26 - DNA, 44 bp, synthetic construct
  source 1..44: mol_type = other DNA
ccaaggtagtcggcaaataattatgacaacttgacggctacatc

SEQ ID NO: 27 - DNA, 46 bp, synthetic construct
  source 1..46: mol_type = other DNA
aattcttgataaattgacatgggagatcctttctcctctttagatc

SEQ ID NO: 28 - DNA, 20 bp, synthetic construct
  source 1..20: mol_type = other DNA
gtctgttattctgtcgccgg

SEQ ID NO: 29 - DNA, 20 bp, synthetic construct
  source 1..20: mol_type = other DNA
aattctgccacagatcaccg

SEQ ID NO: 30 - DNA, 20 bp, synthetic construct
  source 1..20: mol_type = other DNA
tgttcactacgtgaaaggcg

SEQ ID NO: 31 - DNA, 23 bp, synthetic construct
  source 1..23: mol_type = other DNA
tgatgcctggagatccttactcg

SEQ ID NO: 32 - DNA, 27 bp, synthetic construct
  source 1..27: mol_type = other DNA
agatggaaccgtcgttgcagatagtaa

SEQ ID NO: 33 - DNA, 27 bp, synthetic construct
  source 1..27: mol_type = other DNA
tgacttactatctgcaacgacggttcc

SEQ ID NO: 34 - DNA, 27 bp, synthetic construct
  source 1..27: mol_type = other DNA
agatctgcatgacccaaaatcgggacg

SEQ ID NO: 35 - DNA, 27 bp, synthetic construct
  source 1..27: mol_type = other DNA
tgaccgtcccgattttgggtcatgcag

SEQ ID NO: 36 - DNA, 27 bp, synthetic construct
  source 1..27: mol_type = other DNA
agattggatcatcttcgtgaaatctcg

SEQ ID NO: 37 - DNA, 27 bp, synthetic construct
  source 1..27: mol_type = other DNA
tgaccgagatttcacgaagatgatcca

SEQ ID NO: 38 - DNA, 27 bp, synthetic construct
  source 1..27: mol_type = other DNA
agattcgacaacctgcatccacaaggg

SEQ ID NO: 39 - DNA, 27 bp, synthetic construct
  source 1..27: mol_type = other DNA
tgaccccttgtggatgcaggttgtcga

SEQ ID NO: 40 - DNA, 27 bp, synthetic construct
  source 1..27: mol_type = other DNA
agatcgtagacattggatgcccgtgta

SEQ ID NO: 41 - DNA, 27 bp, synthetic construct
  source 1..27: mol_type = other DNA
tgactacacgggcatccaatgtctacg

SEQ ID NO: 42 - DNA, 27 bp, synthetic construct
  source 1..27: mol_type = other DNA
agatcggtcaataaaccatctatgacc

SEQ ID NO: 43 - DNA, 27 bp, synthetic construct
  source 1..27: mol_type = other DNA
tgacggtcatagatggtttattgaccg

SEQ ID NO: 44 - DNA, 46 bp, synthetic construct
  source 1..46: mol_type = other DNA
ccttactcgagtttggatccgccagtttagaccaaaaaaatctatc

SEQ ID NO: 45 - DNA, 21 bp, synthetic construct
  source 1..21: mol_type = other DNA
ctatggtcgaacagctacaac

SEQ ID NO: 46 - DNA, 47 bp, synthetic construct
  source 1..47: mol_type = other DNA
ttgtagctgttcgaccatagtaaaatgatcaaaaaattggcgcggcg

SEQ ID NO: 47 - DNA, 40 bp, synthetic construct
  source 1..40: mol_type = other DNA
tcaaaacacgcacggtgttagccgctgcaatcatcaattg

SEQ ID NO: 48 - DNA, 43 bp, synthetic construct
  source 1..43: mol_type = other DNA
ttgtagctgttcgaccatagcatatcgttatggacggtgatcg

SEQ ID NO: 49 - DNA, 42 bp, synthetic construct
  source 1..42: mol_type = other DNA
tcaaaacacgcacggtgttacgcaaaacctcgacatcactag

SEQ ID NO: 50 - DNA, 46 bp, synthetic construct
  source 1..46: mol_type = other DNA
ttgtagctgttcgaccataggacgaattcttttaatccgggaattg

SEQ ID NO: 51 - DNA, 45 bp, synthetic construct
  source 1..45: mol_type = other DNA
tcaaaacacgcacggtgttagcaatcaattcttcggtctggaaag

SEQ ID NO: 52 - DNA, 42 bp, synthetic construct
  source 1..42: mol_type = other DNA
ttgtagctgttcgaccatagttcgttgtttttgctcgctgtc

SEQ ID NO: 53 - DNA, 47 bp, synthetic construct
  source 1..47: mol_type = other DNA
tcaaaacacgcacggtgttacaaaattggaaaagccgttttaaagaa

SEQ ID NO: 54 - DNA, 41 bp, synthetic construct
  source 1..41: mol_type = other DNA
ttgtagctgttcgaccatagtttgagctcatcaacgatgcc

SEQ ID NO: 55 - DNA, 44 bp, synthetic construct
  source 1..44: mol_type = other DNA
tcaaaacacgcacggtgttagcaataatcttgccagccaatatg

SEQ ID NO: 56 - DNA, 47 bp, synthetic construct
  source 1..47: mol_type = other DNA
tgtagctgttcgaccatagtggcaaagttttctattctagaatcgcc

SEQ ID NO: 57 - DNA, 44 bp, synthetic construct
  source 1..44: mol_type = other DNA
tcaaaacacgcacggtgttatagactcactgatgcccatcttag

SEQ ID NO: 58 - DNA, 42 bp, synthetic construct
  source 1..42: mol_type = other DNA
ccttactcgagtttggatcctgcgagagcgatgtcaaaaacc

SEQ ID NO: 59 - DNA, 44 bp, synthetic construct
  source 1..44: mol_type = other DNA
agcaaatggccaaaactattaaagaacagattccgctcatcaac

SEQ ID NO: 60 - DNA, 24 bp, synthetic construct
  source 1..24: mol_type = other DNA
aatagttttggccatttgcttcac

SEQ ID NO: 61 - DNA, 45 bp, synthetic construct
  source 1..45: mol_type = other DNA
tcaaaacacgcacggtgttaaccaattccgaggcaataagataag

SEQ ID NO: 62 - DNA, 48 bp, synthetic construct
  source 1..48: mol_type = other DNA
ccttactcgagtttggatccggtaggtatcaatccggcttatctattg

SEQ ID NO: 63 - DNA, 28 bp, synthetic construct
  source 1..28: mol_type = other DNA
tttaatcgaaaacgcattacccaattcc

SEQ ID NO: 64 - DNA, 44 bp, synthetic construct
  source 1..44: mol_type = other DNA
gtaatgcgttttcgattaaaaacagccgttattttcagaaaagc

SEQ ID NO: 65 - DNA, 44 bp, synthetic construct
  source 1..44: mol_type = other DNA
tcaaaacacgcacggtgttaaaatgatcgaagcctatgaatccg

SEQ ID NO: 66 - DNA, 38 bp, synthetic construct
  source 1..38: mol_type = other DNA
ccttactcgagtttggatccccgatccgccctatggtc

SEQ ID NO: 67 - DNA, 29 bp, synthetic construct
  source 1..29: mol_type = other DNA
cgattgcttactttcttctctggaatatc

SEQ ID NO: 68 - DNA, 25 bp, synthetic construct
  source 1..25: mol_type = other DNA
aggtgcggtcttgattagccttgaa

SEQ ID NO: 69 - DNA, 40 bp, synthetic construct
  source 1..40: mol_type = other DNA
tcaaaacacgcacggtgttagtgctatccgcttggctctc

SEQ ID NO: 70 - DNA, 47 bp, synthetic construct
  source 1..47: mol_type = other DNA
gagaagaaagtaagcaatcgttaagacccactttcacatttaagttg

SEQ ID NO: 71 - DNA, 44 bp, synthetic construct
  source 1..44: mol_type = other DNA
ggctaatcaagaccgcacctttaattgaataccatcccaccgtc

SEQ ID NO: 72 - DNA, 21 bp, synthetic construct
  source 1..21: mol_type = other DNA
taacaccgtgcgtgttttgac

SEQ ID NO: 73 - DNA, 23 bp, synthetic construct
  source 1..23: mol_type = other DNA
ggatccaaactcgagtaaggatc

SEQ ID NO: 74 - DNA, 21 bp, synthetic construct
  source 1..21: mol_type = other DNA
cgcgaagccattattcgcatc

SEQ ID NO: 75 - DNA, 24 bp, synthetic construct
  source 1..24: mol_type = other DNA
attccgcctgaataggatcagatc

SEQ ID NO: 76 - DNA, 26 bp, synthetic construct
  source 1..26: mol_type = other DNA
attggttagatgatcggaatccgtgc

SEQ ID NO: 77 - DNA, 24 bp, synthetic construct
  source 1..24: mol_type = other DNA
cggctcctgactgttattttctcc

SEQ ID NO: 78 - DNA, 23 bp, synthetic construct
  source 1..23: mol_type = other DNA
aaaagaagggattggcgtttttc

SEQ ID NO: 79 - DNA, 27 bp, synthetic construct
  source 1..27: mol_type = other DNA
cttgttttattgaaagcctgtcatgtc

SEQ ID NO: 80 - DNA, 26 bp, synthetic construct
  source 1..26: mol_type = other DNA
gatccgaaaatttgcaggattagagc

SEQ ID NO: 81 - DNA, 25 bp, synthetic construct
  source 1..25: mol_type = other DNA
gggattaaataccagatgctgatcc

SEQ ID NO: 82 - DNA, 32 bp, synthetic construct
  source 1..32: mol_type = other DNA
atagcaaaagacagccaagttatatataattg

SEQ ID NO: 83 - DNA, 26 bp, synthetic construct
  source 1..26: mol_type = other DNA
tagctgaagaaaccggttgtaaaatc

SEQ ID NO: 84 - DNA, 25 bp, synthetic construct
  source 1..25: mol_type = other DNA
ttatcttatgcctgccagatgatgc

SEQ ID NO: 85 - DNA, 25 bp, synthetic construct
  source 1..25: mol_type = other DNA
gagatgaactacaatggtgtgttcc

SEQ ID NO: 86 - DNA, 21 bp, synthetic construct
  source 1..21: mol_type = other DNA
atccgcttggctctcgaaaag

(19) United States
(12) Patent Application Publication
(10) Pub. No.: US 2026/0234545 A1
(43) Pub. Date: Aug. 13, 2026

(54) ZYMOMONAS MOBILIS CHASSIS CELL AND CONSTRUCTION METHOD THEREFOR AND USE THEREOF

(71) Applicant: Hubei University, Wuhan (CN)

(72) Inventors: Shihui YANG, Wuhan (CN); Yujie ZHANG, Wuhan (CN); Jian LIU, Wuhan (CN); Yunfeng ZHOU, Wuhan (CN); Xiaoyue HU, Wuhan (CN); Binan GENG, Wuhan (CN)

(73) Assignee: Hubei University, Wuhan (CN)

(21) Appl. No.: 19/360,752

(22) Filed: Oct. 16, 2025

(30) Foreign Application Priority Data
Jan. 17, 2025 (CN) 2025100788925

(51) Int. Cl. C12N 1/20; C12N 9/22; C12N 15/10; C12N 15/113; C12N 15/90; C12R 1/01

(52) U.S. Cl. CPC C12N 1/20; C12N 9/226; C12N 15/102; C12N 15/113; C12N 15/902; C12Y 301/11; C12N 2310/20; C12N 2800/101; C12N 2830/34; C12R 2001/01

(57) ABSTRACT

This disclosure belongs to the field of genetic engineering technology, and in particular relates to a Zymomonas mobilis chassis cell, a construction method therefor and use thereof. The chassis cell uses a recombinant Zymomonas mobilis strain with the Cas12a gene integrated into a genome as a starting strain, and integrates the RecET gene into a genome of the recombinant Zymomonas mobilis strain to obtain a chassis cell with improved efficiency of large-fragment gene editing. The present disclosure provides an efficient editing method for directional editing of large fragments of the Zymomonas mobilis genome, which is suitable for the construction of chassis cells requiring frequent gene editing, and greatly reduces the time of gene editing of chassis cells; moreover, the genome of the chassis cell is greatly simplified, which improves the biosynthesis efficiency of biological components and pathways and demonstrates good industrial application value.

### CROSS-REFERENCE TO RELATED APPLICATIONS

The application claims priority to Chinese patent application No. 2025100788925, filed on Jan. 17, 2025, the entire contents of which are incorporated herein by reference.

### SEQUENCE LISTING

The sequence listing xml file submitted herewith, named “WI-US25-31156-P-new.xml”, created on Mar. 23, 2026, and having a file size of 87,251 bytes, is incorporated by reference herein.

### TECHNICAL FIELD

The present disclosure relates to the field of genetic engineering technology, and in particular to a Zymomonas mobilis chassis cell, a construction method therefor and use thereof.

### BACKGROUND

As a frontier field of biotechnology, synthetic biology is rapidly transforming traditional biomanufacturing. Its core lies in constructing efficient cell factories, which are regarded as efficient production units. By utilizing the biosynthetic capacity of cells, genetic engineering, metabolic engineering, and other means are employed to modify and optimize cells, and functional biosynthetic elements, circuits and pathways are introduced to enable them to achieve specific biological functions or mass-produce specific biological products according to predesigned routes. In this process, the chassis cell acts as the host cell for metabolic reactions, responsible for carrying and executing specific biosynthetic tasks, serving as the cornerstone of synthetic biology. For example, when designing a synthetic biological system for producing biofuels, genes encoding enzymes related to biofuel synthesis need to be introduced into the chassis cell to construct a cell factory. The transcription and translation mechanisms within the cell factory will express these genes into corresponding enzymes, which further catalyze a series of chemical reactions in the cell to achieve green and efficient biosynthesis of biofuels.

A chassis cell refers to a genetically simplified basic organism modified by humans, whose genome can be manipulated to facilitate the introduction of functional genetic expression elements. In simple terms, it is a cellular vector that carries and operates the artificially designed biosynthetic system. To enable the chassis cell to better achieve the goals of synthetic biology, it is necessary to transform the chassis cell to ensure benign interaction between the synthetic elements and the host cell. Microbial genome simplification and optimization is an important strategy for constructing excellent chassis cells. Large-scale deletion of non-essential coding and non-coding regions in the genome yields a “minimal genome”. Knocking out redundant genes reduces cellular energy consumption, allowing more energy to be used for product production. Among them, genome large-fragment deletion technology is an important means to achieve genome simplification. Currently, the commonly used genome editing tool CRISPR-Cas has efficient DNA targeting and cleavage efficiency, but the efficiency of large-fragment knockout remains low, especially in microorganisms with low double-strand break repair efficiency. In addition, chassis cells usually involve genetic manipulation and gene editing of multiple genes, so constructing an efficient genetic operation system is of great importance for the efficient transformation of chassis cells.

Zymomonas mobilis is a natural ethanol-producing strain with characteristics such as a small genome, fast metabolic rate, and high ethanol tolerance. It can be used for the production and synthesis of various alcohol and acid products, showing potential to become an excellent industrial chassis cell. At present, two sets of gene editing tools, the endogenous type I-F CRISPR-Cas system and the exogenous CRISPR-Cas12a system, have been constructed in the model strain ZM4 of Zymomonas mobilis, enabling knockout, replacement, and insertion of the strain's genome. However, like most strains, Zymomonas mobilis has low double-strand DNA break recombination efficiency, still making it difficult to achieve large-fragment DNA deletion and replacement. Therefore, it is necessary to develop an efficient large-fragment gene sequence editing system suitable for constructing Zymomonas mobilis chassis cells.

### SUMMARY

In view of the problem of low efficiency of large-fragment gene editing when Zymomonas mobilis is used for chassis cell construction in the prior art, the present disclosure provides a novel Zymomonas mobilis chassis cell, a construction method therefor and use thereof. The present disclosure specifically adopts the following technical solutions:

In a first aspect, the present disclosure provides a Zymomonas mobilis chassis cell; the chassis cell uses Zymomonas mobilis ZMNP-Cas12a with the Cas12a gene integrated at the genomic ZMO0038 locus as a starting strain, integrates the RecET gene into the ZMO0028 locus of the genome of Zymomonas mobilis ZMNP-Cas12a, and the upstream of the RecET gene is linked to a Pₜₑₜ promoter, and the upstream of the Cas12a gene is linked to a P_{BAD} promoter.

Further, the nucleotide sequence of the Cas12a gene is shown in SEQ ID NO.1, the nucleotide sequence of the RecET gene is shown in SEQ CD NO. 2, the nucleotide sequence of the Peₜₑₜ promoter is shown in SEQ ID NO.3, and the nucleotide sequence of the P_{BAD} promoter is shown in SEQ ID NO.4.

In a second aspect, the present disclosure provides a method for constructing the Zymomonas mobilis chassis cell, including the following steps:
obtaining the upstream homologous arm 0038US and downstream homologous arm 0038DS sequences of the genomic ZMO0038 locus, tandemly linking the P_{BAD} promoter and Cas12a gene between the 0038US and 0038DS sequences, and constructing a first homologous recombination plasmid;
obtaining the upstream homologous arm 0028US and downstream homologous arm 0028DS sequences of the genomic ZMO0028 locus, tandemly linking the Pₜₑₜ promoter and RecET gene between the 0028US and 0028DS sequences, and constructing a second homologous recombination plasmid; and
transforming the first homologous recombination plasmid and the second homologous recombination plasmid into Zymomonas mobilis ZMNP-Cas12a respectively, integrating the P_{B}aw promoter and the Cas12a gene into the ZMO0038 locus and integrating the Pₜₑₜ promoter and the RecET gene into the ZMO0028 locus through homologous recombination, and screening to obtain the Zymomonas mobilis chassis cell.

In a third aspect, the present disclosure provides use of the Zymomonas mobilis chassis cell in large-fragment gene editing.

Further, the gene editing is gene knockout, gene replacement, and/or point mutation; the gene sequence length ranges from 9 to 30 kb.

In a fourth aspect, the present disclosure provides a gene editing method for the Zymomonas mobilis chassis cell, including the following steps:
designing guide RNA and donor DNA according to the target sequence to be edited, wherein the donor DNA includes upstream homologous arm and downstream homologous arm sequences of the guide RNA targeting sequence, linking the guide RNA and donor DNA to an expression vector to obtain an editing plasmid; transforming the editing plasmid into the Zymomonas mobilis chassis cell, and culturing positive chassis cells transformed with the editing plasmid.

Advantages and positive effects: The present disclosure provides a recombinant strain in which the Cas12a gene expression is controlled by the P_{BAD} promoter and the RecET gene expression is controlled by the Pₜₑₜ promoter. The strain has high efficiency of large-fragment gene editing and editing plasmid transformation. In the presence of guide RNA (gRNA) and donor DNA, the CRISPR-Cas12a and RecET homologous recombination systems are used to conveniently and accurately perform operations such as knockout, site-directed mutation, and replacement on the target sequence within the size range of 9-30 kb in the recombinant strain. This provides an efficient editing method for the directional editing of the Zymomonas mobilis genome, is suitable for the construction of chassis cells requiring frequent gene editing, and greatly reduces the time of gene editing of chassis cells. Moreover, the genome of the chassis cell is greatly simplified, which improves the biosynthesis efficiency of functional genetic expression elements and increases the yield of target products. The strain is suitable as a chassis cell for synthetic biology and has good industrial application value.

### BRIEF DESCRIPTION OF DRAWINGS

In order to more clearly illustrate the technical solutions in the embodiments of the present disclosure, accompanying drawings used for description of embodiments are described briefly below.

FIG. 1 is a schematic diagram showing the principle of using Zymomonas mobilis chassis cells for gene editing according to an embodiment of the present disclosure;

FIG. 2 is a vector map of homologous recombination plasmids pUC57-Pₜₑₜ-RecET and pUC57-P_{BAD}-RecET according to an embodiment of the present disclosure;

FIG. 3 shows the knockout efficiency of the ZMO0252 gene by the recombinant strains Pt-cas12a_Pt-recET and Pt-cas12a_PB-recET according to an embodiment of the present disclosure, in which, FIG. A is a schematic diagram of the recombinant strains transforming the editing plasmid, FIG. B is a diagram of the transformation efficiency of the editing plasmid in the recombinant strains, and FIG. C is a diagram of the gene editing efficiency of the recombinant strains;

FIG. 4 is a vector map of the homologous recombination plasmid pUC57-P_{BAD}-Cas12a according to an embodiment of the present disclosure;

FIG. 5 shows the knockout efficiency of the ZMO0252 gene by the recombinant strain PB-Cas12a according to an embodiment of the present disclosure, in which, FIG. A is a schematic diagram of the recombinant strain transforming the editing plasmid, FIG. B is a diagram of the transformation efficiency of the editing plasmid in the recombinant strain, and FIG. C is a diagram of the gene editing efficiency of the recombinant strain;

FIG. 6 shows the knockout efficiency of the ZMO0252 gene by different recombinant strains according to an embodiment of the present disclosure, in which, FIG. A is a diagram of the colony PCR amplification results of each recombinant strain, and FIG. B is a diagram of the gene editing efficiency of each recombinant strain;

FIG. 7 shows the knockout efficiency of the recombinant strain PB-cas12a_Pt-recET for different fragment lengths of the ZMO0252-0269 gene cluster according to an embodiment of the present disclosure, in which FIG. A is a diagram showing the principle of gene knockout, FIG. B is a diagram showing the accuracy of the knockout sequence verified by Sanger sequencing, and FIG. C is a diagram showing the editing efficiency of different gene fragment lengths;

FIG. 8 is a diagram of the colony PCR amplification results of the knockout of different fragment lengths of the ZMO0252-0269 gene cluster by the recombinant strain PB-cas12a_Pt-recET according to an embodiment of the present disclosure;

FIG. 9 shows the knockout efficiency of the recombinant strain PB-cas12a_Pt-recET for the ZMO1455-1466 and ZMO0793-0803 gene clusters according to an embodiment of the present disclosure, in which FIG. A is a diagram showing the gene cluster knockout efficiency, and FIGS. B—C are diagrams showing the colony PCR amplification results of the ZMO1455-1466 and ZMO0793-0803 gene clusters, respectively; and

FIG. 10 shows the knock-in efficiency of the recombinant strain PB-cas12a_Pt-recET for the 2,3-butanediol metabolic pathway gene cluster according to an embodiment of the present disclosure, in which FIG. A is a schematic diagram of the gene knock-in site, and FIGS. B—C are diagrams showing the colony PCR amplification results of the recombinant strain PB-cas12a_Pt-recET and the control strain PB-cas12a, respectively.

### DETAILED DESCRIPTION OF THE EMBODIMENTS

In order to make the objectives, technical solutions, and advantages of the present disclosure clearer, the present disclosure is further described in detail below with reference to embodiments of the present disclosure. It should be understood that the specific embodiments described herein are only intended to explain the present disclosure and are not intended to limit the present disclosure. According to the information contained herein, it will be readily apparent to those skilled in the art that various changes may be made to the precise description of the disclosure without departing from the spirit and scope of the appended claims. In order to better understand the present disclosure rather than limit the scope of the present disclosure, all figures representing quantities, percentages, and other numerical values used in this application should be understood as being modified by the word “about” in all cases. Unless otherwise specified, the numerical parameters set forth in the specification and appended claims are approximations that may vary depending upon the desired properties sought to be obtained. Each numerical parameter should at least be construed as being subject to a conventional rounding method. Additionally, it should be noted that, unless otherwise defined, scientific and technical terms used in the context of the present disclosure should have the meanings commonly understood by those of ordinary skill in the art.

The terms “include”, “comprising”, “containing”, “having” and similar words are non-restrictive in meaning, that is, other steps and other components that do not affect the results can be added. The term “and/or” should be regarded as a specific disclosure of each of the two specified features or components with or without the other. For example, “A and/or B” will be deemed to include the following cases: (i) A, (ii) B, and (iii) A and B. The terms “first” and “second” are used for descriptive purposes only and should not be understood as indicating or implying relative importance or relative sequence.

The term “gene” refers to the entire nucleotide sequence required to produce a polypeptide chain or functional RNA. The terms “chimeric gene” or “fusion gene” have the same meaning and refer to any gene that is not a natural gene, including regulatory sequences and coding sequences that do not exist together in nature. Thus, a fusion gene may include regulatory sequences and coding sequences derived from different organisms, or regulatory sequences and coding sequences derived from the same organism but arranged in a manner different from that found in nature.

The term “vector” refers to a self-replicating DNA molecule that transfers exogenous target genes into a host organism, and is often in the form of a circular double-stranded DNA molecule. A vector containing an exogenous gene is a recombinant vector. Typical vectors include plasmids, viruses, phages, cosmids and minichromosomes. Plasmids are the most common form of vectors; accordingly, in the context of the present disclosure, plasmids and vectors can be used interchangeably.

Unless otherwise specified, the terms “Pₜₑₜ”, “Pₜₑₜ promoter”, “Pₜₑₜ operator” and similar words have the same meaning, which include tetR and tetA sequences, indicating that the expression of the promoter is controlled in the presence of the inducer tetracycline. Unless otherwise specified, the terms “Pw)”, “PaAD promoter”, “Pia) operon” and similar words have the same meaning, which include araC and araBAD sequences, indicating that the expression of the promoter is controlled in the presence of the inducer arabinose.

In order to make the above objectives, features, and advantages of the present disclosure more apparent and understandable, specific embodiments of the present disclosure are described in detail below.

In several preliminary studies of the present disclosure, Zymomonas mobilis ZM4 was first used as a starting strain, and the nuclease Cas12a gene and spectinomycin resistance gene from Francisella novicida were integrated into the ZMO0038 locus in the Z. mobilis ZM4 (ATCC31821) genome by homologous recombination. An inducible promoter, tetracycline-inducible promoter Pₜₑₜ, was used to control the expression level of the nuclease to construct the recombinant strain ZM4-Cas12a. The recombinant strain ZM4-Cas12a is based on the CRISPR-Cas12a system and can achieve directional editing of the genome. For the construction method of the recombinant strain ZM4-Cas12a, reference is made to the literature “Shen W, Zhang J, Geng B, et al. Establishment and application of a CRISPR-Cas12a assisted genome-editing system in Zymomonas mobilis[J].Microbial Cell Factories, 2019, 18(1).DO1:10.1186/s12934-019-1219-5.” and the patent “CN110358767A: GENOME EDITING METHOD FOR ZYMOMONAS MOBILIS BASED ON THE CRISPR-CAS12A SYSTEM AND USE THEREOF (publication date: Oct. 22, 2019).”

Further, using ZM4-Cas12a as a starting strain, the four endogenous plasmids pZM32, pZM36, pZM33, and pZM39 of ZM4-Cas12a were eliminated by genetic engineering to obtain the endogenous plasmid-free recombinant strain Zymomonas mobilis ZMNP-Cas12a. The four endogenous plasmids in the ZM4-Cas12a strain are named in order of size: pZM32 (32791 bp. Genbank accession No. CP023678), pZM33 (33006 bp, Genbank accession No. CP023679), pZM36 (36494 bp, Genbank accession No. CP023680), and pZM39 (39266 bp, Genbank accession No.CP023681). The recombinant strain ZMNP-Cas12a has a streamlined genome, which is conducive to reducing energy consumption, and has excellent properties such as high gene transformation efficiency, better tolerance to ethanol fermentation inhibitors, and better ability to utilize secondary mother liquor, making it suitable as a chassis cell for industrial microorganisms. The construction method of the recombinant bacterium ZMNP-Cas12a is described in the patent “CN115806922A: GENETICALLY ENGINEERED STRAIN OF ZYMOMOjAS MOBILIS AND USE THEREOF (publication date: Mar. 17, 2023).”

The genome sequence of Zymomonas mobilis ZM4 used in the present disclosure is shown in Genbank accession number CCA 003054575.

Although the recombinant strain ZMNP-Cas12a provides a versatile genome editing tool for the development of synthetic microbial chassis and has application advantages as chassis cells, its efficiency in editing large-fragment genes or DNA sequences is low.

Based on this, an embodiment of the present disclosure provides a novel recombinant strain of Zymomonas mobilis, which is suitable as a chassis cell for large-fragment gene editing. The chassis cell uses Zymomonas mobilis ZMNP-Cas12a with the Cas12a gene integrated at the genomic ZMO0038 locus as a starting strain, integrates the RecET gene into the ZMO0028 locus of the genome of Zymomonas mobilis ZMNP-Cas12a, and the upstream of the RecET gene is linked to a Pₜₑₜ promoter, and the upstream of the Cas12a gene is linked to a P_{BAD} promoter.

Optionally, the nucleotide sequence of the Cas12a gene is shown in SEQ ID NO.1, the nucleotide sequence of the RecET gene is shown in SEQ ID NO.2, the nucleotide sequence of the Pₜₑₜ promoter is shown in SEQ ID NO.3, and the nucleotide sequence of the P_{BAD} promoter is shown in SEQ ID NO.4.

The RecET homologous recombination system derived from the Escherichia coli Rac phage can effectively induce recombination between linear DNA fragments and bacterial chromosomes. The RecE protein in the RecET homologous recombination system has 5′-3 double-stranded exonuclease activity, which can act on linear fragments to produce 3′-end single-stranded overhangs. The overhangs will be further bound by the single-stranded binding protein RecT in the system to prevent degradation. The double-stranded DNA with the protruding ends can promote homologous recombination of the homologous arms at the target site. In the present disclosure, the RecET system is combined with the previously studied CRISPR-Cas12a system. Guided by the guide RNA (gRNA) in the CRISPR-Cas12a system, the Cas12a nuclease recognizes the target sequence of the guide RNA and cleaves to cause double-strand DNA (dsDNA) breaks. The RecE protein binds to the broken dsDNA ends and rapidly degrades from the 5′ end to form a long 3′ single-strand DNA (ssDNA) overhang. Subsequently, the RecT protein stably binds to the ssDNA to protect the exposed ssDNA from attack by single-strand nucleases and can find the homologous sequence of the ssDNA to achieve gene editing of the target sequence based on homologous recombination. Thus, the CRISPR-Cas12a combined with the RecET homologous recombination system provided by the present disclosure is conducive to promoting the efficient breakage and repair of double-stranded DNA, thereby improving the efficiency of large-fragment editing.

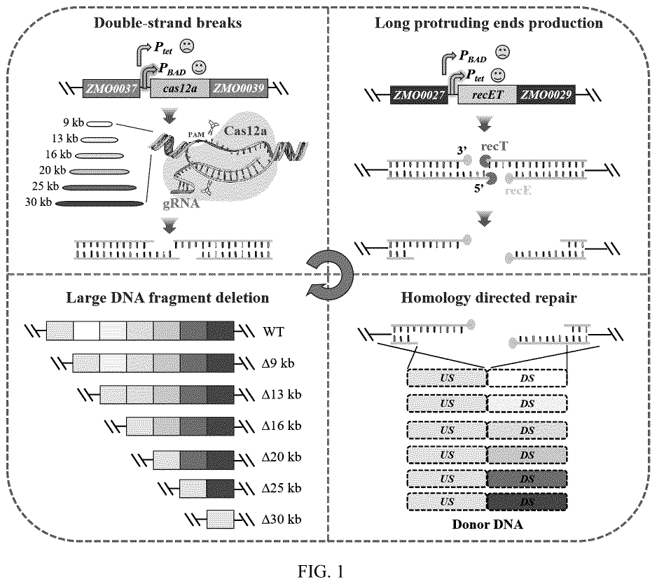

Based on this technical principle (see FIG. 1), the present disclosure uses Zymomonas mobilis ZMNP-Cas12a (Pt-Cas12a) as a starting strain, integrates the RecET gene into the genomic ZMO0028 locus by homologous recombination, and uses two inducible promoters, Pₜₑₜ and P_{BAD}, to control their expression, respectively, to obtain two recombinant strains, Pt-cas12a_Pt-recET and Pt-cas12a_PB-recET. By testing and comparing with the control strain Pₜer-Cas12a, it was found that the Pₜₑₜ promoter-driven RecET expression was more conducive to improving the transformation efficiency of the editing plasmid and the efficiency of large-fragment gene knockout in the recombinant strain. Further, by controlling the expression of the Cas12a gene with Pₜₑₜ and P_{BAD} respectively and optimizing the expression level of Cas12a, it was found that the expression level of Cas12a is crucial for large-fragment gene knockout. Driving Cas12a expression with a more rigorous Pₜₑₜ promoter is more conducive to improving the efficiency of editing plasmid transformation and large-fragment gene knockout in recombinant strains.

Based on the above findings, the present disclosure provides a recombinant strain PB-cas12a_Pt-recET in which the P_{BAD} promoter controls the expression of the Cas12a gene and the Pₜₑₜ promoter controls the expression of the RecET gene. The strain has high efficiency of large-fragment gene editing and transformation of editing plasmid. In the presence of guide RNA (gRNA) and donor DNA (the target gene to be edited into the targeting sequence of the recombinant strain genome, including at least the upstream homologous arm (US) and downstream homologous arm (DS) of the targeting sequence), the CRISPR-Cas12a and RecET homologous recombination systems are used to mediate the recombination of the targeting sequence break site with the homologous arm of the donor DNA. The targeting sequence of the recombinant strain can be easily and accurately replaced with the target gene for operations such as DNA knockout, site-directed mutagenesis and replacement, solving the problem of frequent re-transformation of chassis cells due to the replacement of genetic expression elements with different functions. The present disclosure demonstrated that the recombinant strain of the present disclosure can effectively knock out large gene fragments within the 9-30 kb range, by using site-directed knockout of multiple fragments of different sizes (9 kb, 13 kb, 16 kb, 20 kb, 25 kb, and 30 kb) within the ZMO0252_0269 gene cluster, the ZMO1455-1466 gene cluster (14 kb), and the ZMO0793-0803 gene cluster (15 kb). The knockout efficiencies for DNA fragments of 9-16 kb, 20-25 kb, and 30 kb in the genome were approximately 100%, 80%, and 5%, respectively. In addition, the application of site-directed replacement of the Pₜₑₜ-als Pgap-aldC_bdh (4393 bp) gene cluster in the 2,3-butanediol metabolic pathway showed that the knock-in efficiency of the gene at the target site was approximately 56.25%. It can be seen that the r^{e}combinant strain with the gene editing system of the present disclosure provides an efficient editing method for the directional editing of the Zymomonas mobilis genome. It has high efficiency in large-fragment knockout and can improve the efficiency of large-fragment gene knock-in. It is suitable for the construction of chassis cells that require frequent gene editing, which greatly saves the time of chassis cell gene editing, and can also provide scientific guidance for large-fragment genome editing of other microbial strains. Moreover, the genome of the starting strain of the present disclosure is greatly simplified, which improves the biosynthesis efficiency of functional genetic expression elements and increases the yield of target products. Thus, it is suitable as a chassis cell for synthetic biology and has good industrial application value.

Another embodiment of the present disclosure provides a method for constructing the Zymomonas mobilis chassis cell as described above, including the following steps:
obtaining the upstream homologous arm 0038US and downstream homologous arm 0038DS sequences of the genomic ZMO0038 locus, tandemly linking the P_{BAD} promoter and Cas12a gene between the 0038US and 0038DS sequences, and constructing a first homologous recombination plasmid;
obtaining the upstream homologous arm 0028US and downstream homologous arm 0028DS sequences of the genomic ZMO0028 locus, tandemly linking the Pₜₑₜ promoter and RecET gene between the 0028US and 0028DS sequences, and constructing a second homologous recombination plasmid; and
transforming the first homologous recombination plasmid and the second homologous recombination plasmid into Zymomonas mobilis ZMNP-Cas12a respectively, integrating the P_{BAD} promoter and the Cas12a gene into the ZMO0038 locus and integrating the Pₜₑₜ promoter and the RecET gene into the ZMO0028 locus through homologous recombination, and screening to obtain the Zymomonas mobilis chassis cell.

It should be noted that the transformation order of the first homologous recombination plasmid and the second homologous recombination plasmid is not particularly limited. The first homologous recombination plasmid can be transformed first, the P_{BAD} promoter and the Cas12a gene are connected in series and integrated into the ZMO0038 locus of Zymomonas mobilis ZMNP-Cas12a to obtain the PB-cas12a recombinant strain, and then the second homologous recombination plasmid is transformed, and the Ptet promoter and the RecET gene are connected in series and integrated into the ZMO0028 locus of the PB-cas12a recombinant strain to obtain the target recombinant strain by screening. Alternatively, the second homologous recombination plasmid can be transformed first to obtain the Pt-cas12a_Pt-recET recombinant strain, and then the first homologous recombination plasmid is transformed to obtain the target recombinant strain by screening.

Optionally, to facilitate screening, the first homologous recombination plasmid and the second homologous recombination plasmid carry different resistance marker genes, so that positive clones are screened through the resistance of the recombinant strain to different antibiotics. The selection of resistance markers in the present disclosure is not particularly limited, and conventional resistance genes in the art can be used, including but not limited to kanamycin gene, chloramphenicol gene, spectinomycin gene, tetracycline gene, ampicillin gene, puromycin gene, neomycin gene.

In a typical embodiment, the resistance marker gene of the first homologous recombination plasmid is selected from the spectinomycin gene, and the resistance marker gene of the second homologous recombination plasmid is selected from the chloramphenicol gene.

Optionally, the first homologous recombination plasmid and the second homologous recombination plasmid further include an origin of replication (Ori) sequence, which may be derived from a pUC57 vector. The Ori sequence initiates replication in the plasmid by recruiting replication-related proteins to obtain a large number of homologous recombination plasmids.

In a specific embodiment, the construction of the first homologous recombination plasmid includes the following steps: obtaining the upstream homologous arm 0038US and downstream homologous arm 0038DS sequences of the ZMO0038 locus by PCR amplification, and obtaining the P_{BAD} promoter. Cas12a gene, spectinomycin gene, and Ori sequence of the pUC57 vector by PCR amplification, and connecting the amplified 0038US sequence, Cas12a gene, P_{BAD} promoter, spectinomycin gene, and 0038DS sequence with the Ori sequence through Gibson assembly to obtain the first homologous recombination plasmid. The nucleotide sequences of the 0038US sequence amplification primer pair are shown in SEQ ID NO.20-21, the nucleotide sequences of the 0038DS sequence amplification primer pair are shown in SEQ ID NO.22-23, the nucleotide sequences of the Cas12a gene amplification primer pair are shown in SEQ ID NO.18-19, the nucleotide sequences of the P_{BAD} promoter amplification primer pair are shown in SEQ ID NO.7 and 9, the nucleotide sequences of the spectinomycin gene amplification primer pair are shown in SEQ ID NO.24-25, and the nucleotide sequences of the Ori sequence amplification primer pair are shown in SEQ ID NO.16-17.

In a specific embodiment, the construction of the second homologous recombination plasmid includes the following steps: obtaining the upstream homologous arm 0028US and downstream homologous arm 0028DS sequences of the ZMO0028 locus by PCR amplification, and obtaining the Pₜₑₜ promoter, RecET gene, chloramphenicol gene, and Ori sequence of the pUC57 vector by PCR amplification, and connecting the amplified 0028US sequence, RecET gene, Pₜₑₜ promoter, chloramphenicol gene, and 0028DS sequence with the Ori sequence through Gibson assembly to obtain the second homologous recombination plasmid. The nucleotide sequences of the 0028US sequence amplification primer pair are shown in SEQ ID NO.10-11, the nucleotide sequences of the 0028DS sequence amplification primer pair are shown in SEQ ID NO.12-13, the nucleotide sequences of the RecET gene amplification primer pair are shown in SEQ ID NO.5-6, the nucleotide sequences of the Pₜₑₜ promoter amplification primer pair are shown in SEQ ID NO.7-8, the nucleotide sequences of the chloramphenicol gene amplification primer pair are shown in SEQ ID NO.14-15, and the nucleotide sequences of the Ori sequence amplification primer pair are shown in SEQ ID NO.16-17.

An embodiment of the present disclosure provides use of the aforementioned Zymomonas mobilis chassis cell in large-fragment gene editing, especially for large-fragment gene editing with a sequence length ranging from 9 to 30 kb, more preferably 9 to 25 kb.

The aforementioned gene editing includes but is not limited to gene knockout, gene replacement, and point mutation, which are realized based on the structural form of the exogenous donor DNA. Specifically, when the donor DNA only includes the upstream homologous arm (US) and downstream homologous arm (DS) of the guide RNA (gRNA) targeting sequence, the targeting sequence can be deleted (see FIG. 1 for the principle). When the target gene includes not only the upstream and downstream homology arm sequences of the targeting sequence, but also the target gene to be knocked in between the upstream and downstream homology arm sequences, the target gene can replace the targeting sequence, thereby realizing the operations of large-fragment gene replacement or point mutation.

The present disclosure also provides a gene editing method for a Zymomonas mobilis chassis cell, including the following steps:
designing guide RNA and donor DNA according to the target sequence to be edited, wherein the donor DNA comprises upstream homologous arm and downstream homologous arm sequences of the guide RNA targeting sequence, linking the guide RNA and donor DNA to an expression vector to obtain an editing plasmid; transforming the editing plasmid into the Zymomonas mobilis chassis cell, and culturing the chassis cells transformed with the editing plasmid, to achieve gene editing of the targeting sequence through homologous recombination technology.

Optionally, the expression vector is artificially modified pEZ15A, and the modification process thereof can be found in the patent “CN110358767A”. Specifically, pEZ15a is used as a vector backbone, into which the following sequence is inserted: TTGACAGCTAGCTCAGTCCTAGGTATAATGCTAGCAATTTCTACTCTTGTAGATGGAGAC CGAGGTCTCA. The underlined wavy line indicates the constitutive promoter PJ23119, the underlined solid straight line indicates the 19nt repeat sequence, and the underlined dotted straight line indicates two Bsa I restriction sites, which are the insertion sites for gRNA.

After the gRNA primer pair with Bsa I restriction sites at the upstream is retreated, it is ligated to the pEZ15A vector digested with Bsa I, then the ligated pEZ15A vector is amplified and ligated with the donor DNA to obtain the editing plasmid.

In the context of the present disclosure, upstream refers to the 5′ end of a gene, and downstream refers to the 3′ end of a gene, with upstream to downstream being from the 5′ end to the 3′ end.

The present disclosure is further described below in conjunction with specific embodiments. The experimental methods in the following embodiments that do not specify specific conditions are usually carried out under conventional conditions, such as those described in the Molecular Cloning Laboratory Manual (Fourth Edition) published by Cold Spring Harbor Laboratory, or those recommended by the manufacturer.

### EXAMPLE

1. Introduction of the RecET System into ZMNP-Cas12a and Determination of the Optimal Promoter

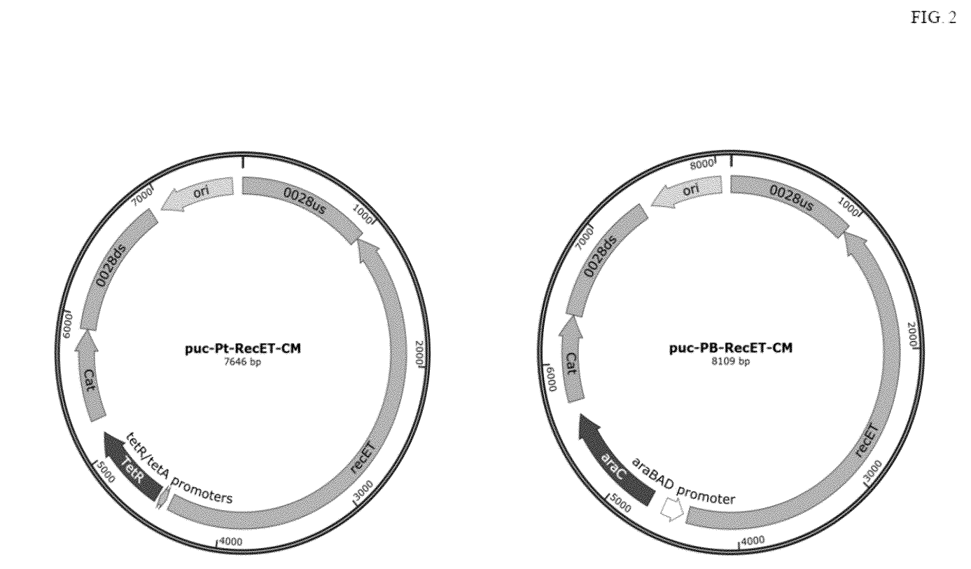

Using Zymomonas mobilis ZMNP-Cas12a (Pt-Cas12a) as the starting strain, the homologous recombination plasmids pUC57-Ptet-RecET (vector map shown in the left diagram of FIG. 2) and pUC57-P_{BAD}-RecET (vector map shown in the right diagram of FIG. 2) were respectively transformed into the strain ZMNP-Cas12a, and the RecET was integrated into the genome ZMO0028 locus through homologous recombination, and the expression of RecET was controlled by two inducible promoters Pₜₑₜ and P_{BAD}, respectively, to obtain the recombinant strains Pt-cas12a_Pt-recET and Pt-cas12a_PB-recET. By testing and comparing with the control strain Pₜₑₜ-Cas12a, the promoter corresponding to the RecET expression with the highest editing plasmid transformation efficiency and the highest large fragment knockout efficiency was determined.

Zymomonas mobilis ZM4 (ATCC31821) was used as the starting strain. The Cas12a gene and spectinomycin gene were integrated into the ZMO0038 gene locus of its genome, and the tetracycline-inducible promoter Pₜₑₜ was operably connected upstream of Cas12a to control the expression level of the nuclease, obtaining the recombinant strain ZM4-Cas12a. The four endogenous plasmids of ZM4-Cas12a, pZM32 (32791 bp, Genbank accession No. CP023678), pZM33 (33006 bp, Genbank accession No. CP023679), pZM36 (36494 bp, Genbank accession No. CP023680) and pZM39 (39266 bp, Genbank accession No. CP023681) were further eliminated to obtain the recombinant strain Zymomonas mobilis ZMNP-Cas12a.

1.1. Construction of Homologous Recombination Plasmids pUC57-Pₜₑₜ-RecET and pUC57-P_{BAD}-RecET

In the embodiment of the present disclosure, the upstream homologous sequence (US) and the downstream homologous sequence (DS) of the ZMO0028 locus of the ZM4 genome were amplified, and the fusion sequence (Pₜₑₜ-RecET or P_{BAD}-RecET) of the operon containing the Pw promoter or P_{BAD} promoter and the RecET gene was placed between the US and DS sequences. The fusion sequence Pₜₑₜ-RecET or P_{BAD}-RecET was integrated into the corresponding locus of the genome by homologous recombination. At the same time, in order to facilitate screening, a resistance marker screening gene, chloramphenicol Cat gene, was added. In addition, to facilitate the transformation of the fusion gene sequence, Pₜₑₜ-RecET or P_{BAD}-RecET and Cat gene as well as US and DS sequences were ligated with the backbone sequence high-copy replicon Ori (replication origin) of the pUC57 vector to form circularized homologous recombination plasmids pUC57-Pₜₑₜ-RecET and pUC57-P_{BAD}-RecET. The gene and primer sequences used to construct the RecET gene homologous recombination plasmids are shown in Table 1. The construction process is as follows:

TABLE 1
Genes and primer sequences used to construct homologous recombination
plasmids in the present disclosure

| Sequence | |
|---|---|
| name | Sequence (5′-3′) |
| Cas12a | atgtcaatttatcaagaatttgtgaacaaatatagcctgagcaaaaccct |
| | gcgttttgaactgattccgcagggtaaaaccctggaaaacattaaagcac |
| | gtggtctgattctggatgatgaaaaacgtgccaaagactacaaaaaagcc |
| | aaacaaatcatcgataaataccaccagttcttcatcgaagaaattctgag |
| | cagcgtttgcattagcgaagatctgctgcagaattattccgacgtttatt |
| | tcaaactgaaaaaaagcgacgatgataacctgcagaaagatttcaaaagc |
| | gccaaagataccatcaaaaaacaaattagcgagtatatcaaagacagcga |
| | gaaattcaaaaacctgttcaaccagaatctgatcgatgccaaaaaaggtc |
| | aagaaagcgatctgatcctgtggctgaaacagagcaagataatggcatcg |
| | aactgtttaaagccaacagcgatattaccgatattgatgaagcactggaa |
| | atcatcaaaagctttaaaggttggaccacctactttaaaggctttcacga |
| | aaatcgcaaaaacgtgtatagcagcaatgatattccgaccagcattatct |
| | atcgcatcgttgatgataatctgcctaaatttctggaaaataaagccaaa |
| | tatgaaagcctgaaagacaaagcaccggaagcaattaactatgagcagat |
| | tcaaaaaagatctggccgaagaactgacctttgacattgattacaaaacc |
| | agcgaagttaaccagcgtgtttttagcctggatgaagtttttgaaattgc |
| | caacttcaacaactacctgaatcagagcggtatcaccaaattcaatacca |
| | ttatcggtggcaaattcgtgaatggcgaaaataccaaacgcaaaggcatc |
| | aacgaatacattaatctgtatagccagcagattaacgataaaacgctgaa |
| | aaaatacaaaatgagcgtgctgttcaxacaaattctgtcagataccgaaa |
| | gcaaaagcttcgtgattgacaaactggaagatgatagtgatgttgttacc |
| | accatgcagagcttttatgaacaaatcgcagcgtttaaaaccgtggaaga |
| | gaaatccattaaagaaaccctgagcctgctgtttgatgatctgaaagcac |
| | agaaactggacctgtccaaaatctaccttcaaaaacgataaatccctgac |
| | cgatctgagccagcaggttttcgatgattatagcgttattggcaccgcag |
| | ttctggaatatatcacacagcagattgcaccgaaaaatctggataatccg |
| | agcaaaaaagaacaagagctgatcgccaaaaaaaccgagaaagcgaaata |
| | tctgagcctggaaacaattaaactggccctggaagaatttaacaaacacc |
| | gcgacattgataaacagtgccgctttgaagaaatcctggcaaattttgca |
| | gcaatcccgatgatctttgatgaaattgcgcagaataaagataacctggc |
| | acagatcagcatcaaatatcagaatcagggaaaaaaagacctgctgcaag |
| | caagtgccgaagatgatgttaaagcgattaaagatctgctggatcagacc |
| | aataacctgctgcataaactgaaaatctttcacattagccagagcgagga |
| | taaagcgaacattctggataaagatgagcacttctatctggtgtttgaag |
| | agtgttattttgagctggcaaatattgtgccgctgtataacaaaatccgc |
| | aactatattacccagaaaccgtatagcgacgaaaaattcaaactgaactt |
| | tgagaatagcaccctggccaatggttgggataaaaacaaagaaccggata |
| | ataccgccatcctgttcattaaagatgataaatactatctgggcgtgatg |
| | aacaaaaaaaacaacaaaatcttcgatcgataaagccatcaaagagaata |
| | aaggcgaaggttacaaaaaaatcgtgtacaaactgctgcctggtgcgaat |
| | aaaatgctgccgaaagtgttttttagcgccaaatccatcaaattctataa |
| | cccgagcgaagatattctgcgtattcgtaatcatagcacccataccaaaa |
| | atggtagtccgcagaaaggctatgaaaaattcgagttcaacattgaggat |
| | tgccgcaaattcatcgacttctacaaacagtccattagcaaacatccgga |
| | atggaaagactttggttttcgttttagcgatacccagcgctataacagca |
| | ttgatgaattttatcgcgaagtggaaaaccagggctataaactgacattt |
| | gaaaacatcagcgagagctatattgatagcgttgtgaatcagggtaaact |
| | gtacctgtttcagatctataacaaagactttagcgcctatagcaaaggtc |
| | gtccgaatctgcataccctgtattggaaagcactgttcgatgaacgtaat |
| | ctgcaggatgttgtctacaaactgaatggtgaagcagaactgttttatcg |
| | caaacagagtatcccgaaaaaaatcacccatccggcaaaagaagcaatcg |
| | cgaacaaaaacaaagataacccgaaaaaagaaagcgtgttcgagtatgat |
| | ctgatcaaagataaacgcttcaccgaagataaattctttttccattgccc |
| | gatcaccatcaactttaaaagcagcggtgcgaacaaattcaacgatgaaa |
| | tcaatctgctgctgaaagaaaaagccaacgatgttcatattctgagcatt |
| | gatcgtggtgaacgtcatctggcctattacaccctggttgatggtaaagg |
| | caatattatcaaacaggacaccttcaacattatcggcaatgatcgtatga |
| | aaaccaactaccatgataaactggcagccattgaaaaagatcgtgatagc |
| | gcacgtaaagattggaaaaaaatcaacaacattaaagaaatgaaagaagg |
| | ctacctgagccaggttgttcatgaaatcgccaaactggtgattgaatata |
| | atgccattgtggtgttcgaggatctgaacttcggtttcaaacgtggtcgt |
| | ttcaaagttgagaaacaggtgtatcaaaaactggaaaaaatgctgatcga |
| | aaaactgaattacctggtgttcaaagacaacgaattcgataaaaccggtg |
| | gtgttctgcgtgcatatcagctgaccgcaccttttgaaaccttcaaaaaa |
| | atgggtaaacagaccggcatcatctattatgttccggcaggttttacctc |
| | caaaatttgtccggttaccggctttgttaatcagctgtatccgaaatatg |
| | agagcgttagcaaaagccaagagtttttcagcaaatttgataaaatctgc |
| | tataacctggacaaaggctactttgaattcagctttgactataaaaactt |
| | tggcgataaagcagccaaaggcaaatggaccattgcaagctttggtagcc |
| | gtctgattaactttcgtaacagcgacaaaaaccataactgggatacccgt |
| | gaagtttatccgaccaaagagctggaaaaactgctgaaagattacagcat |
| | tgaatatggtcatggcgaatgtattaaagccgcaatttgtggtgagtccg |
| | acaaaaaattctttgcaaaactgaccagcgtgctgaataccattctgcag |
| | atgcgtaatagcaaaaccggcaccgaactggattatctgattagtccggt |
| | tgcagatgtgaacggcaattttttcgatagccgtcaggctccgaaaaata |
| | tgccgcaggatgcagatgcaaatggtgcctatcatattggcctgaaaggt |
| | ctgatgctgctgggtcgcattaaaaacaatcaagaaggcaaaaaactgaa |
| | cctggtgatcaaaaacgaagagtattttgagttcgtgcagaataggaata |
| | actaa |
| | (see SEQ ID NO. 1) |
| RecET | atgagcacaaaaccactcttcctgttacggaaagcgaaaaaatcatccgg |
| | tgaacctgacgtcgtcctgtgggcaagcaacgattttgaatcgacctgtg |
| | ccactctggactacctgatcgttaagtcaggtaaaaaactgagcagctat |
| | tttaaagctgttgccacgaattttcctgtcgttaatgacctgcccgctga |
| | aggtgagatcgattttacctggagtgaacgctatcaactcagcaaagact |
| | ccatgacatgggaactaaaaccgggagcagcaccagacaacgctcactat |
| | caaggcaataccaaegtcaacggegaagacatgactgagattgaggagaa |
| | tatgctactcccaatttctggccaggaactgcccattcgttggcttgctc |
| | aacacggcagcgaaaaaccggtaacgcacgtttcacgcgacggactccag |
| | gcattacacattgctcgggctgaagaactaccggctgttactgccctggc |
| | tgtttcccacaaaaccagcctgctcgacccgctggaaattcgcgaactcc |
| | acaaactggttcgtgacactgacaaagttttccctaatcctggtaattca |
| | aacctgggactgataactgcttttttcgaagcatacctgaacgctgacta |
| | caccgatcgaggactgctgacaaaagagtggatgaagggtaatcgtgttt |
| | cacacatcactcgcacggcttccggtgctaatgctggcggcggaaacctc |
| | accgatcgcggcgaaggtttcgtacacgatctgacgtcactggcgcgcga |
| | cgtagccactggcgtactggcccgttcaatggatctggacatctataacc |
| | ttcatccggcacacgctaaacgcattgaggaaattatcgctgaaaataaa |
| | ccgcccttttctgttttccgcgacaaattcatcaccatgcctggcgggct |
| | ggattattcccgcgccatcgtggttgcgtccgtaaaagaagcaccaattg |
| | ggatcgaggtcatccccgcgcacgtcactgaatatctgaacaaagttgta |
| | ctgactgaaaccgatcatgccaaccctgatccggaaatcgtggatattgc |
| | ctgcggtcgctcctctgccccgatgccgcagcgagtaacagaagaaggaa |
| | aacaggatgatgaagaaaaaccgcaaccatctggaacaacggcagttgaa |
| | cagggagaggctgaaacaatggaaccggacgcaactgaacatcatcagga |
| | cacgcagccgctggatgctcagtcacaggtaaattctgttgatgcgaaat |
| | atcaggaactgcgggcagaactccatgaagcccggaaaaacattccatca |
| | aaaaatcctgtcgatgacgataaattgcttgctgcatcacgtggtgaatt |
| | tgttgacggaattagcgacccgaacgatccgaaatgggtaaaggggatcc |
| | agactcgcgattgtgtgtaccagaaccagccagaaacggaaaaaaccagc |
| | ccagatatgaatcaacctgagccagtagtgcaacaggaaccggaaatagc |
| | ctgcaatgcctgcggccagactggcggggataactgccctgactgtggtg |
| | cggtgatgggcgacgcaacataccaggaaacattcgatgaagagagtcag |
| | gttgaagctaaggaaaatgatccggaggaaatggaaggcgctgaacatcc |
| | gcacaatgagaatgctggcagcgatccgcatcgcgattgcagtgatgaaa |
| | ctggcgaagtcgcagatcccgtaatcgtagaagacatagagccaggtatt |
| | tattacggaatttcgaatgagaattaccacgcgggtcccggtatcagtaa |
| | gtctcagctcgatgacattgctgatactccggcactatatttgtggcgta |
| | aaatgcccccgtggacaccacaaagacaaaaacgctcgatttaggaactg |
| | ctttccactgccgggtacttgaaccggaagaattcagtaaccgctttatc |
| | gtagcacctgaatttaaccgccgtacaaacgccggaaaagaagaagagaa |
| | agcgtttctgatggaatgcgcaagcacaggaaaaacggttatcactgcgg |
| | aagaaggccggaaaattgaactcatgtatcaaagcgttatggctttgccg |
| | ctggggcaatggcttgttgaaagcgccggacacgctgaatcatcaattta |
| | ctgggaagatcctgaaacaggaattttgtgtcggtgccgtccggacaaaa |
| | ttatccctgaatttcactggatcatggacgtgaaaactacggcggatatt |
| | caacgattcaaaaccgcttattacgactaccgctatcacgttcaggatgc |
| | attctacagtgacggttatgaagcacagtttggagtgcagccaactttcg |
| | tttttctggttgccagcacaactattgaatgcggacgttatccggttgaa |
| | attttcatgatgggcgaagaagcaaaactggcaggtcaacaggaatatca |
| | ccgcaatctgcgaaccctgtctgactgcctgaataccgatgaatggccag |
| | ctattaagacattatcactgccccgctgggctaaggaatatgcaaatgac |
| | taagcaaccaccaatcgcaaaagccgatctgcaaaaaactcagggaaacc |
| | gtgcaccagcagcagttaaaaatagcgacgtgattagttttattaaccag |
| | ccatcaatgaaagagcaactggcagcagctcttccacgccatatgacggc |
| | tgaacgtatgatccgtatcgccaccacagaaattcgtaaagttccggcgt |
| | taggaaactgtgacactatgagttttgtcagtgcgatcgtacagtgttca |
| | cagctcggacttgagccaggtagcgccctcggtcatgcatatttactgcc |
| | ttttggtaataaaaacgaaaagagcggtaaaaagaacgttcagctaatca |
| | ttggctatcgcggcatgattgatctggctcgccgttctggtcaaatcgcc |
| | agcctgtcagcccgtgttgtccgtgaaggtgacgagtttagcttcgcctt |
| | gatgaaaagttaatacaccgcccgggagaaaacgaagatgccccggttac |
| | ccacgtctatgctgtcgcaagactgaaagacggaggtactcagtttgaag |
| | ttatgacgcgcaaacagattgagctggtgcgcagcctgagtaaagcttgg |
| | taataacgggccgtgtaactcactgggaagaaatggcaaagaaaacggct |
| | attcgtcgcctgttcaaatatttgcccgtatcaattgagatccagcgtgc |
| | agtatcaatggatgaaaaggaaccactgacaatcgatcctgcagattcct |
| | ctgtattaaccggggaatacagtgtaatcgataattcagaggaataa |
| | (see SEQ ID NO. 2) |
| Pₜₑₜ | ttaagacccactttcacatttaagttgtttttctaatccgcatatgatca |
| | attcaaggtcgaataagaaggctggctctgcaccttggtgatcaaataat |
| | tcgatagcttgtcgtaataatggcggcatactatcagtagtaggtgtttc |
| | cctttcttctttagcgacttgatgctcttgatcttccaatacgcaaccta |
| | aagtaaaatgccccacagcgctgagtgcatataatgcattctctagtgaa |
| | aaaccttgttggcataaaaaggctaattgattttcgagagtttcatactg |
| | tttttctgtaggccgtgtacctaaatgtacttttgctccatcgcgatgac |
| | ttagtaaagcacatctaaaacttttagcgttattacgtaaaaaatcttgc |
| | cagctttccccttctaaagggcaaaagtgagtatggtgcctatctaacat |
| | ctcaatggctaagcggcgtcgagcaaagcccgcttattttttacatgcca |
| | atacaatgtaggctgctctacacctagcttctgggcgagtttacggcttg |
| | ttaaaccttcgattccgacctcattaagcagctctaatgcgctgttaatc |
| | actttacttttatctaatctagacataattcctaagttgacactctatcg |
| | ttgatagagttattttaccactccctatcagtgatagagaaaagtattca |
| | aatgatctaaagaggagaaaggatctccc |
| | (see SEQ ID NO. 3) |
| P_{BAD} | Ttatgacaacttgacggctacatcattcactttttcttcacaaccggcac |
| | ggaactcgctcgggctggccccggtgcattttttaaatacccgcgagaaa |
| | tagagttgatcgtcaaaaccaacattgcgaccgacggtggcgataggcat |
| | ccgggtggtgctcaaaagcagcttcgcctggctgatacgttggtcctcgc |
| | gccagcttaagacgctaatccctaactgctggcggaaaagatgtgacaga |
| | cgcgacggcgacaagcaaacatgctgtgcgacgctggcgatatcaaaatt |
| | gctgtctgccaggtgatcgctgatgtactgacaagcctcgcgtacccgat |
| | tatccatcggtggatggagcgactcgttaatcgcttccatgcgccgcagt |
| | aacaattgctcaagcagatttatcgccagcagctccgaatagcgcccttc |
| | cccttgcccggcgttaatgatttgcccaaacaggtcgctgaaatgcggct |
| | ggtgcgcttcatccgggcgaaagaaccccgtattggcaaatattgacggc |
| | cagttaagccattcatgccagtaggcgcgcggacgaaagtaaacccactg |
| | gtgataccattcgcgagcctccggatgacgaccgtagtgatgaatctctc |
| | ctgggggaacagcaaaatatcacccggtcggcaaacaaattctcgtccct |
| | gatttttcaccaccccctgaccgcgaatggtgagattgagaatataacct |
| | ttcattcccagcggtcggtcgataaaaaaatcgagataaccgttggcctc |
| | aatcggcgttaaacccgccaccagatgggcattaaacgagtatcccggca |
| | gcaggggatcattttgcgcttcagccataaaccaattgtccatattgcat |
| | cagacattgccgtcactgcgtcttttactggctcttctcgctaaccaaac |
| | cggtaaccccgcttattaaaagcattctgtaacaaagcgggaccaaagcc |
| | atgacaaaaacgcgtaacaaaagtgtctataatcacggcagaaaagtcca |
| | cattgattatttgcacggcgtcacactttgctatgccatagcatttttat |
| | ccataagattagcggatcctacctgacgctttttatcgcaactctctact |
| | gtttctccataagtattcaaatgatctaaagaggagaaaggatctccc |
| | (SEE SEQ ID NO. 4) |
| RecET-F | TTAAGTAAAAGGCTCGATATTATTCCTCTGAATTATCGATTACACTGTATTCCC |
| | (see SEQ ID NO. 5) |
| RecET-R | AAGAGGAGAAAGGATCTCCCATGAGCACAAAACCACTCTTCCTG (see SEQ ID |
| | NO. 6) |
| Pₜₑₜ/P_{BAD} | GGGAGATCCTTTCTCCTCTTTAG (see SEQ ID NO. 7) |
| F | |
| Pₜₑₜ-R | AGCTGTCAAACATGAGAATTTTAAGACCCACTTTCACATTTAAGTTG (see SEQ ID |
| | NO. 8) |
| P_{BAD}-R | AGCTGTCAAACATGAGAATTTTATGACAACTTGACGGCTACATC (see SEQ ID |
| | NO. 9) |
| 0028US- | TAAGGGATTTTGGTCATGAGCGGATOCTGCTTTGGATTTG (see SEQ ID NO. 10) |
| F | |
| 0028US- | TATCGAGCCTTTTACTTAAAATAATCTATCGAAAG (see SEQ ID NO. 11) |
| R | |
| 0028DS- | GCTTTTTGAAAAAGCCGTTTCCTGTATTG (see SEQ ID NO. 12) |
| F | |
| 0028DS-R | TCACATGTTCTTTCCTGCGTTTACCAGCATGTTTTATCAGGAATCG (see SEQ ID |
| | NO. 13) |
| Cat-F | AATTCTCATGTTTGACAGCTTATCATCG (see SEQ ID NO. 14) |
| Cat-R | AAACGGCTTTTTCAAAAAGCTTACGCCCCGCCCTGCCACTC (see SEQ ID NO. 15) |
| Ori-F | ACGCAGGAAAGAACATGTGAGC (see SEQ ID NO. 16) |
| Ori-R | CTCATGACCAAAATCCCTTAACGTG (see SEQ ID NO. 17) |
| Cas12a-F | AAGAGGAGAAAGGATCTCCCATGTCAATTTATCAAGAATTTGTGAACAAATATA |
| | G (see SEQ ID NO. 18) |
| Cas12a-R | CCGTCTGGCGTCGGGCGTGATAAAACGAAAGGCCCAGTCTTTC (see SEQ ID |
| | NO. 19) |
| 0038US- | TAAGGGATTTTGGTCATGAGTTAGGCGAGAAGGGAAAGGGCAAG (see SEQ ID |
| F | NO. 20) |
| 0038US- | TCGTTAAATATTCAGATAGACGGAGATAATAAACGGG (see SEQ ID NO. 21) |
| R | |
| 0038DS-F | TCACGCCCGACGCCAGAC (see SEQ ID NO. 22) |
| 0038DS-R | TCACATGTTCTTTCCTGCGTCACCCTCTGGTGATTGTCGATATC (see SEQ ID |
| | NO. 23) |
| Spe-F | TCTATCTGAATATTTAACGAAATTCTCATGTTTGACAGCTTATCATCG (see SEQ ID |
| | NO. 24) |
| Spe-R | TTATTTGCCGACTACCTTGGTG (see SEQ ID NO. 25) |
| P_{BAD}-F1 | CCAAGGTAGTCGGCAAATAATTATGACAACTTGACGGCTACATC (see SEQ ID |
| | NO. 26) |
| P_{BAD}-R1 | AATTCTTGATAAATTGACATGGGAGATCCTTTCTCCTCTTTAGATC (see SEQ ID |
| | NO. 27) |

(1) Extraction of ZM4 genome: 2 mL of overnight cultured ZM4 bacteria was collected and the genome was extracted using a bacterial genome extraction kit (purchased from Beijing TransGen Biotech Co., Ltd., Catalog No. EE10101R30722-V2R137).
(2) The ZMO0028 US sequence (abbreviated as 0028US) was amplified using the 0028US-F/R primer pair with the ZM4 genome as a template; the ZMO0028 DS sequence (abbreviated as 0028DS) was amplified using the 0028DS-F/R primer pair with the ZM4 genome as a template; the chloramphenicol gene Cat was amplified using the Cat-F/R primer pair with the pEZ15A plasmid as a template; the Ori sequence was amplified using the Ori-F/R primer pair with the pUC57 plasmid as a template; the RecET gene was amplified using the RecET-F/R primer pair with the Escherichia coli MG1655 genome as a template; the Pₜₑₜ operon (including tetR and tetA) and P_{BAD} operon (including araC and ara_{BAD}) were amplified using the Pₜₑₜ/P_{BAD}-F and Pₜₑₜ-R primer pairs, and the Pw/Pean-F and P_{BAD}-R primer pairs with the p39-Pₜₑₜ-araC-T7P-Cm plasmid as a template. The construction method of the p39-Pₜₑₜ-araC-T7P-Cm plasmid is described in the patent “CN114774453A”.
(3) The amplified 0028US, 0028DS, Pₜₑₜ operon, RecET, and Cat gene sequences, and the vector backbone Ori sequence were added to 5 μL of 2×CE Mix (purchased from Nanjing Vazyme Biotech Co., Ltd., product number 027E4210KD3) and mixed. Each sequence was added at a concentration of 0.03pM, and ddH2O was added to make up to 10 μL. The fragments were connected through a one-step Gibson assembly reaction of the clone enzyme to obtain the pUC57-Pₜₑₜ-RecET plasmid. The amplified 0028US, 0028DS, P_{BAD} operon, RecET and Cat gene sequences and the vector backbone Ori sequence were ligated based on the same reaction system to obtain the pUC57-P_{BAD}-RecET plasmid.
(4) The homologous recombination plasmids pUC57-Pₜₑₜ-RecET and pUC57-P_{BAD}-RecET were respectively transformed into Escherichia coli DH5a cell competent cells, and positive clones were verified by LB plates containing chloramphenicol resistance. The plasmids were extracted from the positive clones after overnight culture. The plasmid extraction was carried out according to the standard steps of the plasmid extraction kit (purchased from Trelief Bio, product number 1N7012490010213).

### 1.2. Construction of Recombinant Strains Pt-Cas12a_Pt-recET and Pt-Cas12a_PB-recET

Transformation of Zymomonas mobilis ZMNP-Cas12a:100 μL of the frozen strain of Zymomonas mobilis ZMNP-Cas12a was inoculated into a cryovial containing 1 mL of RMG5 liquid medium and statically cultured in a 30° C. incubator to activate the strain. When turbidity was observed, the culture was transferred to a 250 mL blue-cap bottle containing 200 mL of RMG5 liquid medium to make the initial OD₆₀₀ₙₘ within the range of 0.025-0.3, and statically cultured in a 30° C. incubator. When OD₆₀₀ₙₘ exceeded 0.3, the bacterial cells were collected by centrifugation at 100 rpm at room temperature, then washed once with sterile water, twice with 10% glycerol. Finally, the bacterial cells were slowly resuspended with 1-2 mL of 10% glycerol to obtain ZMNP-Cas12a competent cells, which were dispensed into 1.5 mL EP tubes at 55 μL of competent cells per tube.

500 ng of the homologous recombination plasmid was added to a 1.5 mL EP tube containing 55 μL of competent cells, gently mixed, and transferred to a 1 mm electroporation cuvette. The electroporator program was set as: 2000, capacitance: 25 μF, voltage: 1.6 KV. The electroporation cuvette was placed in the electroporator for electroporation. After electroporation, 1 mL of RMG5 liquid medium was immediately added, mixed, transferred to a sterile EP tube, sealed with a sealing film, and incubated in a 30° C. constant temperature incubator for 4-6 hours. Then, 100 μL of the culture was evenly spread onto an RMG5 plate containing 100 μg/mL chloramphenicol (Cm resistance), and cultured upside down in a 30° C. incubator for 2 days.

Single colonies were selected for colony PCR verification. The primer pair for verification of the recombinant strains Pt-cas12a_Pt-recET and Pt-cas12a_PB-recET was RecET-YZ-F/R. The colony PCR system (10 μL) included: 0.4 μL of RecET-YZ-F (10 μM), 0.4 μL of RecET-YZ-R (10 μM), 5 μL of 2×T5 Super PCR Mix (Tsingke), Template (single colony dissolved in 10 μL of ddH2O), and 3.2 μL of ddH₂O. The colony PCR program was set as: pre-denaturation at 98′C for 3 min; denaturation at 98′C for 10 s, annealing at 55° C. for 10 s, extension at 72° C. for 70 s, for 29 cycles; extension at 72° C. for 3 min, and holding at 16′C. The positive clones verified by colony PCR were further verified by sequencing, to obtain the target strains Pt-cas12a_Pt-recET and Pt-cas12a_PB-recET with the Pₜₑₜ-recET and P_{BAD}-recET expression cassettes integrated into the genomic ZMO0028 locus.

### 1.3. Detection of Gene Editing Efficiency of Recombinant Strains Pt-Cas12a_Pt-recET and Pt-Cas12a_PB-recET

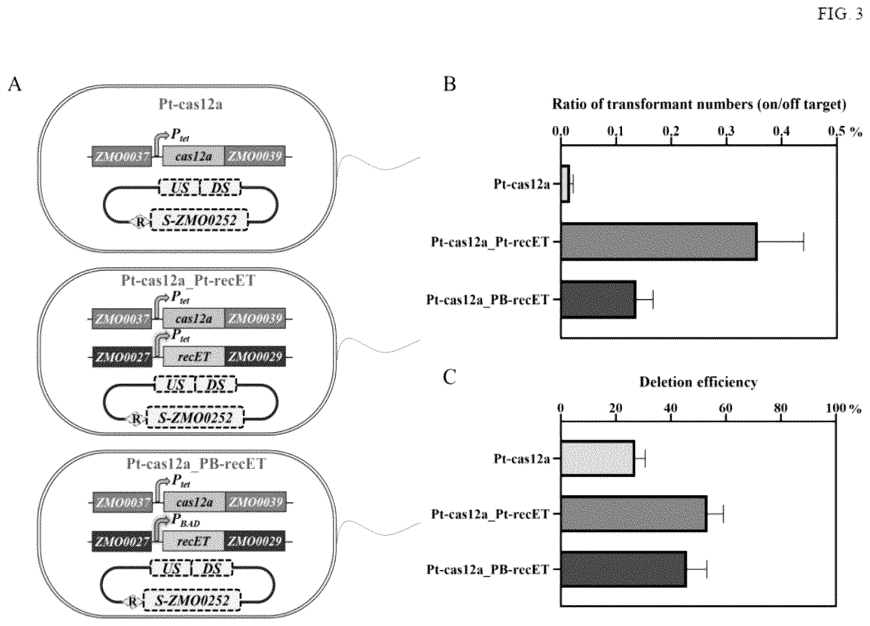

The editing plasmid pKO-ZMO0252 with ZMO0252 knockout (~9 kb) was transformed into the Pt-cas12a_Pt-recET and Pt-cas12a_PB-recET strains and the control strain Pt-Cas12a (see FIG. 3A), respectively. The transformation efficiency of the editing plasmid and the efficiency of large-fragment gene editing of each strain were compared and determined. The pKO-ZMO0252 plasmid contains gRNA targeting the ZMO0252 gene and upstream and downstream homologous sequences of the ZMO0252 gene. The gRNA is used to guide the nuclease Cas12a to cut the target site. The upstream and downstream homologous sequences of ZMO0252 are used as donor DNA to perform homologous recombination with the upstream and downstream of the ZMO0252 gene in the cell genome to achieve the deletion of ZMO0252. The preparation method was described in detail in the subsequent section.

The transformation efficiency of the editing plasmid is defined as: The number of colonies on the Kanamycin-resistant RMG2 plate after the editing plasmid was transformed into the recombinant strain/the number of colonies on the Kanamycin-resistant RMG2 plate after the empty plasmid pEZ15A was transformed into the recombinant strain. Method for determination: 200 ng of the editing plasmid and empty plasmid were added to 1.5 mL EP tubes containing 55 μL of competent cells, respectively, mixed gently, and then transferred to a 1 mm electroporation cuvette. The electroporator program was set as: 200Ω, capacitance: 25 μF, voltage: 1.6 KV. The electroporation cuvette was placed in the electroporator for electroporation. After electroporation, 1 mL of RMG5 liquid medium was immediately added, mixed, transferred to a sterile EP tube, sealed with a sealing film, and incubated in a 30° C. constant temperature incubator for 4 hours. Then 100 μL of the bacterial solution was evenly spread on an RMG2+Kana plate (300 μg mL⁻¹ kanamycin). The plate was sealed with a sealing film and placed in a 30° C. incubator upside down. After culturing for two days, the colonies on the plate were counted.

Large fragment editing efficiency is defined as: The number of colonies with successful knockout of ZMO0252 verified by PCR/the total number of colonies verified by PCR; The determination methods include: A single colony of the recombinant strain transformed with the editing plasmid on the RMG2 Kana plate was randomly selected and the ZMO0252 gene was amplified using the 0252-check-F/R primer pair. The colony PCR system and PCR procedure were the same as above. If a band of approximately 1 kb was detected, it was confirmed that the gene was knocked out. Definition of the large fragment editing efficiency: the number of colonies with successful knockout of ZMO0252 verified by PCR/the total number of colonies verified by PCR. Method of determination: A single colony of the recombinant strain transformed with the editing plasmid on the RMG2Kana plate was randomly selected and the ZMO0252 gene was amplified using the 0252-check-F/R primer pair. The colony PCR system and PCR program were the same as above. If a band of approximately 1 kb was detected, it was confirmed that the gene was knocked out.

FIG. 3 shows the results of large-fragment gene knockout efficiency detection in the recombinant strains Pt-cas12a_Pt-recET and Pt-cas12a_PB-recET, in which, FIG. A is a schematic diagram of the transformation of the recombinant strain into the editing plasmid pKO-ZMO0252, FIG. B is a transformation efficiency (Ratio of transformant numbers) diagram of the transformation of the recombinant strain into the editing plasmid pKO-ZMO0252, on-target represents the number of colonies on the Kana-resistant RMG2 plate after the editing plasmid is transformed into the recombinant strain, off-target represents the number of colonies on the Kana-resistant RMG2 plate after the empty plasmid pEZ15A is transformed into the recombinant strain, and FIG. C is a large-fragment editing efficiency diagram of the transformation of the recombinant strain into the editing plasmid pKO-ZMO0252. The results show that the transformation efficiency of the editing plasmid of the control strain ZNMP-Cas12a (Pt-Cas12a) was 0.01±0.01%, while the transformation efficiencies of the recombinant strains Pt-cas12a_Pt-recET and Pt-cas12a_PB-recET were 0.36±0.07% and 0.13±0.03%, respectively, indicating that the introduction of the RecET system can improve the transformation efficiency of the editing plasmid, and the RecET expression level driven by the Pw promoter is more conducive to improving the transformation efficiency of the editing plasmid. The knockout efficiency of ZMO0252 in Pt-cas12a_Pt-recET was 53.33±4.71%, and the knockout efficiency of ZMO0252 in Pt-cas12a_PB-recET was 45.83±5.89%. The knockout efficiency of ZMO0252 in the control strain Pt-Cas12a was 27.08±2.95%, indicating that the introduction of the RecET system can improve the knockout efficiency of large genes, and the expression level of RecET driven by the Pₜₑₜ promoter is more conducive to improving the knockout efficiency of large-fragment genes.

### 2. Optimization of the Zymomonas Mobilis Cas12a Promoter

Although the introduction of the RecET system improved the transformation efficiency of the editing plasmid and the efficiency of large-fragment gene editing, the knockout efficiency of the ZMO0252 gene was still less than 60%, which was possibly caused by the excessive leakage expression of Pw-driven Cas12a, producing toxicity to cells. Therefore, the Cas12a promoter was replaced with a more rigorous inducible promoter P_{BAD}. The homologous recombination plasmid pUC57-P_{BAD}-Cas12a was transformed into the Pt-Cas12a strain, and the recombinant strain PB-Cas12a was obtained by homologous recombination.

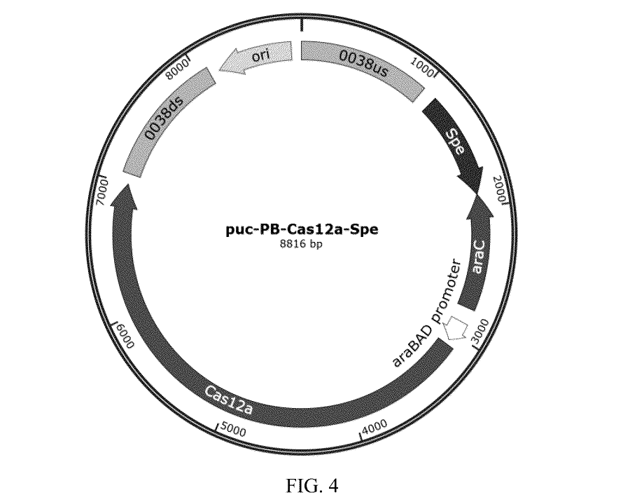

The construction process of pUC57-P_{BAD}-Cas12a was as follows: Primers with homology to pUC57-Ori and the spectinomycin gene Spe were designed. The ZMO0038 US sequence (abbreviated as 0038US) was amplified using the 0038US-F/R primer pair with the ZM4 genome as a template, and the ZMO0038 DS sequence (abbreviated as 0038DS) was amplified using the 0038DS-F/R primer pair; the spectinomycin gene Spe was amplified using the Spe-F/R primer pair with the pEZ15A plasmid as a template; the Ori sequence was amplified using the Ori-F/R primer pair with the pUC57 plasmid as a template; the P_{BAD} operon (including araC and araBAD) was amplified using the P_{BAD}-F1/R1 primer pair with the p39-Pₜₑₜ-araC-T7P-Cm plasmid containing the P_{BAD} promoter as a template. The plasmid construction method was described in the patent “CN114774453A”. The Cas12a gene was amplified using the primer pair Cas12a-F/R with the genome of the strain ZM4-Cas12a as a template. The amplified 0038US, 0038DS, P_{BAD} operon, Cas12a gene, Spe gene sequences, and the vector backbone Ori sequence were added to 5 μL of 2×CE Mix, at a concentration of 0.03 pM for each sequence, then ddH₂O was added to make up to 10 μL. The fragments were connected by Gibson assembly to obtain the pUC57-P_{BAD}-Cas12a plasmid. The vector map is shown in FIG. 4. The aforementioned primer sequences are shown in Table 1.

The homologous recombination plasmid pUC57-P_{BAD}-Cas12a was transformed into Escherichia coli DH5α cell competent cells, and positive clones were verified by LB plates containing Spe resistance and colony PCR, using the verification primer pair Cas12a-YZ-F/R. The positive clones were cultured overnight, the plasmids were extracted, transformed into Zymomonas mobilis ZMNP-Cas12a to obtain the recombinant strain PB-Cas12a.

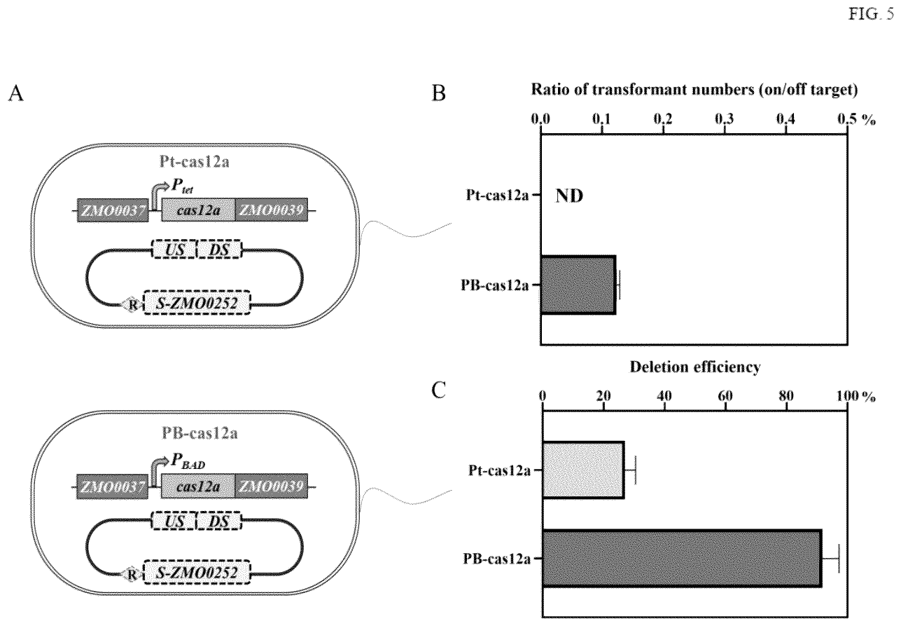

The editing plasmid pKO-ZMO0252 was transformed into the recombinant strain PB-Cas12a to determine the transformation efficiency of the editing plasmid and the efficiency of large-fragment gene editing of the recombinant strain PB-Cas12a. FIG. 5 shows the detection results of large-fragment gene knockout efficiency in the recombinant strain PB-Cas12a, in which FIG. A is a schematic diagram of the recombinant strain transforming the editing plasmid pKO-ZMO0252, FIG. B is a diagram of the transformation efficiency of the recombinant strain transforming the editing plasmid pKO-ZMO0252, and FIG. C is a diagram of the large-fragment editing efficiency diagram of the recombinant strain transformation editing plasmid pKO-ZMO0252. The results showed that the transformation efficiency of the control strain Pt-Cas12a was almost 0, and the transformation efficiency of the recombinant strain PB-cas12a was 12.37±0.36%, indicating that reducing the expression level of Cas12a can improve the transformation efficiency of the editing plasmid, and the expression level of Cas12a driven by the P_{BAD} promoter is more conducive to improving the transformation efficiency of the editing plasmid. The knockout efficiency of ZMO0252 in PB-cas12a was 91.55±4.05%, while the knockout efficiency of ZMO0252 in the control strain Pt-Cas12a was 27.00 ±2.83%. The efficiency of large-fragment gene editing of the PB-cas12a strain was 3 times higher than that of the Pt-Cas12a strain, indicating that the expression level of Cas12a is crucial for large-fragment gene knockout, and the expression level of Cas12a driven by the P_{BAD} promoter is more conducive to improving the efficiency of large-fragment gene knockout.

### 3. Construction of Zymomonas Mobilis PB-Cas12a_Pt-recET

A series of recombinant strains with different expression levels of Cas12a and RecET were constructed, including single-system expression strains Pt-cas12a, PB-cas12a, and Pt-recET, and dual-system expression strains Pt-cas12a_Pt-recET, Pt-cas12a_PB-recET, and PB-cas12a_Pt-recET. The recombinant strain Pt-recET was obtained by electroporation of pUC57-P_{W}-RecET using Zymomonas mobilis ZMNP as the host. The integration of the Pw-recET expression cassette into the ZMO0028 locus of the ZMNP genome was verified by the RecET-YZ-F/R primer pair and sequencing. The recombinant strain PB-cas12a_Pt-recET was obtained by electroporation of pUC57-Pan-Cas12a using Pt-cas12a_Pt-recET as a host, or by transformation of pUC57-Pm-RecET using PB-cas12a as a host, which was not described again here.

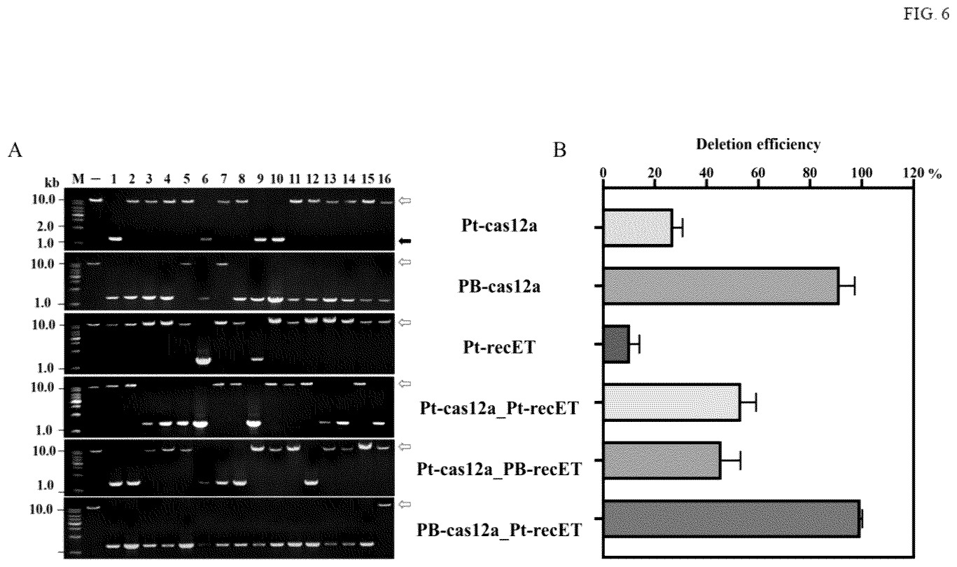

The pKO-ZMO0252 was electroporated into each recombinant strain, and 16 positive colonies were randomly selected. The knockout efficiency of the ZMO0252 gene in different strains was verified by colony PCR using the 0252-check-FIR primer pair. The results are shown in FIG. 6, in which. FIG. A is a diagram of the colony PCR amplification results of each recombinant strain transformed with the editing plasmid pKO-ZMO0252, lane M represents a molecular marker, lane—represents PCR amplification using the ZMNP genome as a template, lanes 1-16 represent 16 positive colonies; FIG. B is a diagram of large-fragment editing efficiencies of each recombinant strain transformed with the editing plasmid pKO-ZMO0252. The results showed that the knockout efficiencies of ZMO0252 in Pt-cas12a, PB-cas12a, Pt-recET, Pt-cas12a_Pt-recET, Pt-cas12a_PB-recET, and PB-cas12a_Pt-recET are 4/16, 13/16, 2/16, 8/16, 6/16, and 15/16, respectively. Among them, the large-fragment knockout efficiency of PB-cas12a_Pt-recET was the highest, approximately 98.53±2.07%.

4. Testing of Knockout/Insertion Efficiency of Large DNA Fragments of Different Lengths by Zymomonas mobilis PB-Cas12a_Pt-recET

In order to determine the knockout efficiency of PB-cas12a_Pt-recET for large DNA fragments of different lengths, PB-cas12a was used as the control strain, the ZMO0252-ZMO0269 gene range was selected, and the editing plasmids pKO-ZMO0252, pKO-ZMO0252_0254, pKO-ZMO0252_0256, pKO-ZMO0252_0262, pKO-ZMO0252_0266, and pKO-ZMO0252_0269 were constructed to knock out ZMO0252 (9 kb), ZMO0252_0254 (13 kb), ZMO0252_0256 (16 kb), ZMO0252_0262 (20 kb), ZMO0252_0266 (25 kb), and ZMO0252_0269 (30 kb) for knockout efficiency test. The PB-cas12a_Pt-recET strain was further used to knock out the transporter/secretion protein gene clusters ZMO1455-1466 (14 kb) and ZMO0793-0803 (15 kb), and the editing plasmids pEZKO-1455_1466 and pEZKO-0793_0803 were constructed. In addition, in order to test the knock-in efficiency of large fragments in PB-cas12a_Pt-recET, the 2,3-butanediol metabolic pathway Pt-als Pgap-aldC_bdh was selected to knock into the genome ZMO1650 position to construct the editing plasmid pEZTH-2,3-BDO-1650 targeting ZMO1650.

In the embodiments of the present disclosure, the genome editing method of Zymomonas mobilis based on the CRISPR-Cas12a system in patent CN110358767A was utilized. The editing plasmid used pEZ15a as the vector backbone (kanamycin (Cm) resistance), and the constitutive promoter PJ23119 (TTGACAGCTAGCTCAGTCCTAGGTATAATGCTAGC), a 19nt repeat sequence (AATTTCTACTCTTGTAGAT) and two Bsa I restriction sites (GGAGACCGAGGTCTCA) were connected in series and assembled into the pEZ15a vector. The guide RNA sequence was inserted between the two Bsa I restriction sites to form a guide RNA expression unit to guide the nuclease Cas12a to cut the target site. The guide RNA was obtained by annealing a guide RNA primer pair with a Bsa I restriction site upstream. The upstream primer structure was 5′-AGAT+(target sequence)-3′, and the downstream primer structure was 5′-TGAC (complementary sequence of the target sequence)-3′. The annealed primer pair was ligated with the pEZ15a vector digested with Bsa I to obtain an editing plasmid containing the CRISPR expression unit. The editing plasmid also included donor DNA, which served as a template for homologous recombination repair to edit the guide RNA targeting sequence. The specific construction method of the relevant editing plasmids was as follows:
(1) Primer design: The 23 bp downstream of the TTTN sequence of the PAM locus in the ZMO0252-0269 gene cluster, ZMO1455-1466 gene cluster, ZMO0793-0803 gene cluster, and ZMO1650 was selected as the targeting sequence for the guide RNA in the constructed target plasmid to direct nuclease cleavage at the target site. The guide RNA primer pair for the ZMO0252, ZMO0252-0254, and ZMO0252-0256 knockout plasmids was 252-gR-F/R; the guide RNA primer pair for the ZMO0252-0262 knockout plasmid was 0254-gR-F/R; the guide RNA primer pair for the ZMO0252-0266 and ZMO0252-0269 knockout plasmids was 0256-gR-F/R; the guide RNA primer pair for the ZMO1455-1466 knockout plasmids was 1455-1466-gR-F/R; the guide RNA primer pair for the ZMO0793-0803 knockout plasmids was 07934)803-gR-F/R; and the guide RNA primer pair for the ZMO1650 replacement plasmids was 1650-gR-F/R.
(2) Ligation of guide RNA to the vector: First, the vector was linearized using the restriction endonuclease Bsa I. Then, the guide RNA primer pairs were annealed (1 μL of each 10 μM primer was mixed with water to make up 10 μL, denatured at 95° C. for 5 min, and then cooled to room temperature for use). The annealed product was ligated with the linearized vector using T4 DNA ligase. The ligation system (50 μL) included: 2 μL of annealed oligonucleotide, 2 μg of linearized vector, 0.5 μL of T4 DNA ligase, 5 μL of T4 ligase buffer, and ddH₂0 to make up 50 μL. Ligation was performed at 22° C. for 3 h. The ligation product was then transformed into the E. coli cloning strain DH5α by conventional methods for plasmid construction. Recombinants were screened by colony PCR and verified by sequencing.
(3) Ligation of donor DNA to the vector: The vector ligated by T4 DNA ligase in the previous step was amplified reversely using the primer PEZ-FK-F/R. The upstream and downstream 700 bp homologous arms of the ZMO0252-0269, ZMO1455-1466, ZMO0793-0803, and ZMO1650 gene clusters were amplified from the ZM4 genome using primer pairs. The upstream and downstream homologous arm amplification primer pairs for ZMO0252 were 0252-US-F/R and 0252-DS-F/R; for ZMO0252-0254, ZMO0252-0256. ZMO0252-0262, ZMO0252-0266, and ZMO0252-0269, the upstream homologous arm amplification primer pair was 0252-US-FIR, and the downstream homologous arm amplification primer pairs were 0254-DS-F/R, 0256-DS-F/R, 0262-DS-F/R, 0266-DS-F/R, and 0269-DS-F/R, respectively; for ZMO1455-1466, the upstream and downstream homologous arm amplification primer pairs were 1455-1466-US-F/R and 1455-1466-DS-F/R; for ZMO0793-0803, the upstream and downstream homologous arm amplification primer pairs were 0793-0803-US-F/R and 0793-0803-DS-F/R; and for the 2,3-BDO synthesis pathway knocked into ZMO1650, the upstream and downstream homologous arm amplification primer pairs were 1650-US-F/R and 1650-DS-F/R. The 2,3-BDO synthesis pathway genes were amplified from the first editing plasmid p12r-1650-BDO of the patent “CN117778291A” using the BDO-F/R primer pair. Fragments were ligated by Gibson assembly, transformed into E. coli DH5αcompetent cells. Positive clones were verified by PCR and the editing plasmids were extracted after overnight culture.

The upstream primer used to verify the ZMO0252-0269 gene cluster was 0252-check-F, and the downstream primers were 0252-check-R, 0254-check-R, 0256-check-R, 0262-check-R, 0266-check-, and 0269-check-R, respectively. The primer pair used to verify the knockout of the ZMO1455-1466 gene cluster was 1455-1466-check-F/R, the primer pair used to verify the knockout of the ZMO0793-0803 gene cluster was 0793-0803-check-FR, and the primer pair used to verify the knock-in of the ZMO1650 in the 2,3-BDO synthesis pathway was BDO-in-F/R. The primer sequences mentioned above are shown in Table 2.

TABLE 2
Guide RNA and donor DNA amplification primers used for constructing editing
plasmids in this example

| Sequence Name | Sequence (5'-3') |
|---|---|
| RecET-YZ-F | GTCTGTTATTCTGTCGCCGG (see SEQ ID NO. 28) |
| RecET-YZ-R | AATTCTGCCACAGATCACCG (see SEQ ID NO. 29) |
| Cas12a-YZ-F | TGTTCACTACGTGAAAGGCG (see SEQ ID NO. 30) |
| Cas12a-YZ-R | TGATGCCTGGAGATCCTTACTCG (see SEQ ID NO. 31) |
| 0252-gR-F | AGATGGAACCGTCGTTGCAGATAGTAA (see SEQ ID NO. 32) |
| 0252-gR-R | TGACTTACTATCTGCAACGACGGTTCC (see SEQ ID NO. 33) |
| 0254-gR-F | AGATCTGCATGACCCAAAATCGGGACG (see SEQ ID NO. 34) |
| 0254-gR-R | TGACCGTCCCGATTTTGGGTCATGCAG (see SEQ ID NO. 35) |
| 0256-gR-F | AGATTGGATCATCTTCGTGAAATCTCG (see SEQ ID NO. 36) |
| 0256-gR-R | TGACCGAGATTTCACGAAGATGATCCA (see SEQ ID NO. 37) |
| 1455-1466-gR-F | AGATTCGACAACCTGCATCCACAAGGG (see SEQ ID NO. 38) |
| 1455-1466-gR-R | TGACCCCTTGTGGATGCAGGTTGTCGA (see SEQ ID NO. 39) |
| 0793-0803-gR-F | AGATCGTAGACATTGGATGCCCGTGTA (see SEQ ID NO. 40) |
| 0793-0803-gR-R | TGACTACACGGGCATCCAATGTCTACG (see SEQ ID NO. 41) |
| 1650-gR-F | AGATCGGTCAATAAACCATCTATGACC (see SEQ ID NO. 42) |
| 1650-gR-R | TGACGGTCATAGATGGTTTATTGACCG (see SEQ ID NO. 43) |
| 0252-US-F | CCTTACTCGAGTTTGGATCCGCCAGTTTAGACCAAAAAAATCTATC |
| | (see SEQ ID NO. 44) |
| 0252-US-R | CTATGGTCGAACAGCTACAAC (see SEQ ID NO. 45) |
| 0252-DS-F | TTGTAGCTGTTCGACCATAGTAAAATGATCAAAAAATTGGCGCGGCG |
| | (see SEQ ID NO. 46) |
| 0252-DS-R | TCAAAACACGCACGGTGTTAGCCGCTGCAATCATCAATTG (see SEQ |
| | ID NO. 47) |
| 0254-DS-F | TTGTAGCTGTTCGACCATAGCATATCGTTATGGACGGTGATCG (see |
| | SEQ ID NO. 48) |
| 0254-DS-R | TCAAAACACGCACGGTGTTACGCAAAACCTCGACATCACTAG (see |
| | SEQ ID NO. 49) |
| 0256-DS-F | TTGTAGCTGTTCGACCATAGGACGAATTCTTTTAATCCGGGAATTG |
| | (see SEQ ID NO. 50) |
| 0256-DS-R | TCAAAACACGCACGGTGTTAGCAATCAATTCTTCGGTCTGGAAAG |
| | (see SEQ ID NO. 51) |
| 0262-DS-F | TTGTAGCTGTTCGACCATAGTTCGTTGTTTTTGCTCGCTGTC (see SEQ |
| | ID NO. 52) |
| 0262-DS-R | TCAAAACACGCACGGTGTTACAAAATTGGAAAAGCCGTTTTAAAGA |
| | A (see SEQ ID NO. 53) |
| 0266-DS-F | TTGTAGCTGTTCGACCATAGTTTGAGCTCATCAACGATGCC (see SEQ |
| | ID NO. 54) |
| 0266-DS-R | TCAAAACACGCACGGTGTTAGCAATAATCTTGCCAGCCAATATG (see |
| | SEQ ID NO. 55) |
| 0269-DS-F | TGTAGCTGTTCGACCATAGTGGCAAAGTTTTCTATTCTAGAATCGCC |
| | (see SEQ ID NO. 56) |
| 0269-DS-R | TCAAAACACGCACGGTGTTATAGACTCACTGATGCCCATCTTAG (see |
| | SEQ ID NO. 57) |
| 1455-1466-US-F | CCTTACTCGAGTTTGGATCCTGCGAGAGCGATGTCAAAAACC (see |
| | SEQ ID NO. 58) |
| 1455-1466-US-R | AGCAAATGGCCAAAACTATTAAAGAACAGATTCCGCTCATCAAC (see |
| | SEQ ID NO. 59) |
| 1455-1466-DS-F | AATAGITTTGGCCATTTGCTTCAC (see SEQ ID NO. 60) |
| 1455-1466-DS-R | TCAAAACACGCACGGTGTTAACCAATTCCGAGGCAATAAGATAAG |
| | (see SEQ ID NO. 61) |
| 0793-0803-US-F | CCTTACTCGAGTTTGGATCCGGTAGGTATCAATCCGGCTTATCTATTG |
| | (see SEQ ID NO. 62) |
| 0793-0803-US-R | TTTAATCGAAAACGCATTACCCAATTCC (see SEQ ID NO. 63) |
| 0793-0803-DS-F | GTAATGCGTTTTCGATTAAAAACAGCCGTTATTTTCAGAAAAGC (see |
| | SEQ ID NO. 64) |
| 0793-0803-DS-R | TCAAAACACGCACGGTGTTAAAATGATCGAAGCCTATGAATCCG (see |
| | SEQ ID NO. 65) |
| 1650-US-F | CCTTACTCGAGTTTGGATCCCCGATCCGCCCTATGGTC (see SEQ ID |
| | NO. 66) |
| 1650-US-R | CGATTGCTTACTTTCTTCTCTGGAATATC (see SEQ ID NO. 67) |
| 1650-DS-F | AGGTGCGGTCTTGATTAGCCTTGAA (see SEQ ID NO. 68) |
| 1650-DS-R | TCAAAACACGCACGGTGTTAGTGCTATCCGCTTGGCTCTC (see SEQ ID |
| | NO. 69) |
| BDO-F | GAGAAGAAAGTAAGCAATCGTTAAGACCCACTTTCACATTTAAGTTG |
| | (see SEQ ID NO. 70) |
| BDO-R | GGCTAATCAAGACCCCACCTTTAATTGAATACCATCCCACCGTC (see |
| | SEQ ID NO. 71) |
| PEZ-FK-F | TAACACCGTGCGTGTTTTGAC (see SEQ ID NO. 72) |
| PEZ-FK-R | GGATCCAAACTCGAGTAAGGATC (see SEQ ID NO. 73) |
| 0252-check-F | CGCGAAGCCATTATTCGCATC (see SEQ ID NO. 74) |
| 0252-check-R | ATTCCGCCTGAATAGGATCAGATC (see SEQ ID NO. 75) |
| 0254-check-R | ATTGGTTAGATGATCGGAATCCGTGC (see SEQ ID NO. 76) |
| 0256-check-R | CGGCTCCTGACTGTTATTTTCTCC (see SEQ ID NO. 77) |
| 0262-check-R | AAAAGAAGGGATTGGCGTTTTTC (see SEQ ID NO. 78) |
| 0266-check-R | CTTGTTTTATTGAAAGCCTGTCATGTC (see SEQ ID NO. 79) |
| 0269-check-R | GATCCGAAAATTTGCAGGATTAGAGC (see SEQ ID NO. 80) |
| 1455-1466- | GGGATTAAATACCAGATGCTGATCC (see SEQ ID NO. 81) |
| check-F | |
| 1455-1466- | ATAGCAAAAGACAGCCAAGTTATATATAATTG (see SEQ ID NO. 82) |
| check-R | |
| 0793-0803- | TAGCTGAAGAAACCOGTTGTAAAATC (see SEQ ID NO. 83) |
| check-F | |
| 0793-0803- | TTATCTTATGCCTGCCAGATGATGC (see SEQ ID NO. 84) |
| check-R | |
| BDO-in-F | GAGATGAACTACAATGGTGTGTTCC (see SEQ ID NO. 85) |
| BDO-in-R | ATCCGCTTGGCTCTCGAAAAG (see SEQ ID NO. 86) |

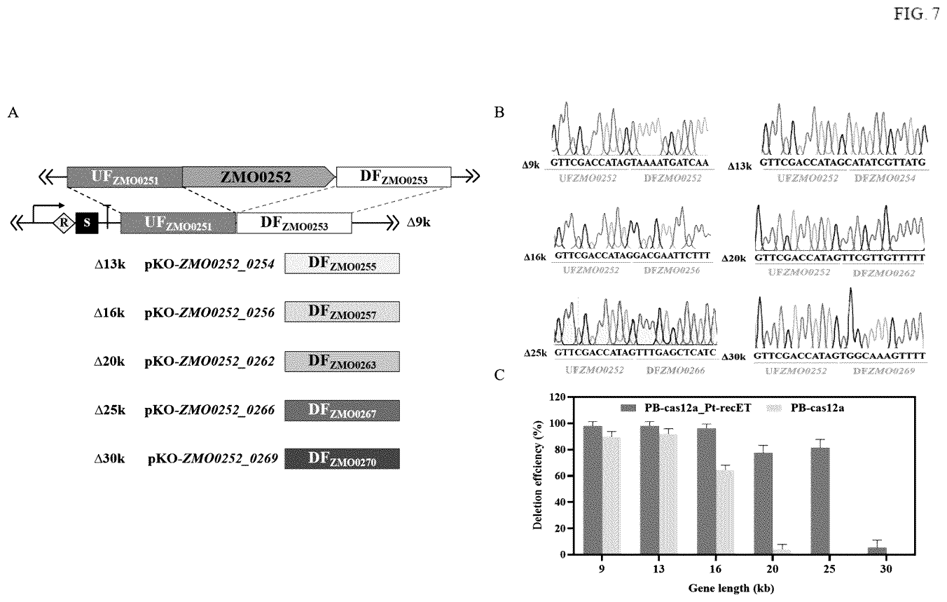

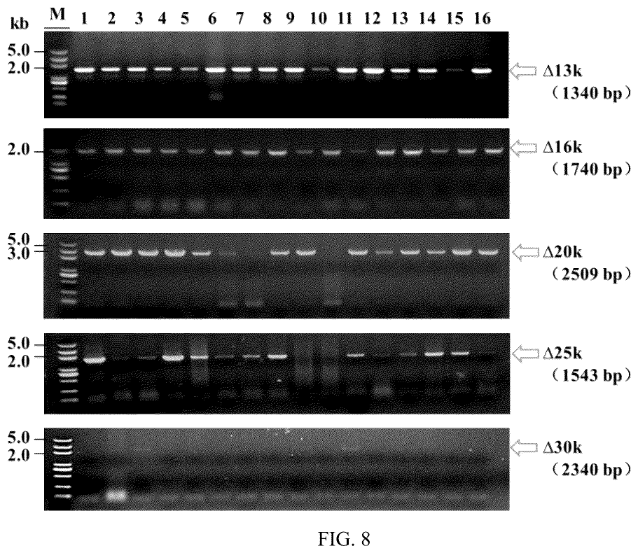

FIG. 7 showed the knockout of the ZMO0252-0269 gene cluster by PB-cas12a and PB-cas12a_Pt-recET strains, in which, FIG. A was a diagram showing the principle of knockout of the ZMO0252-0269 gene cluster, FIG. B was a diagram showing the accuracy of the knockout sequence verified by Sanger sequencing, FIG. C was a diagram showing the editing efficiency of different gene fragment lengths of the ZMO0252-0269 gene cluster. FIG. 8 showed the knockout results of different fragment lengths of the ZMO0252-0269 gene cluster by the recombinant strain PB-cas12a_Pt-recET, where lane M represented a molecular marker and lanes 1-16 represented 16 positive colonies. The results showed that the knockout efficiency of 9-16 kb large-fragment genes in PB-cas12a_Pt-recET was approximately 100%, that of 20-25 kb was approximately 80%, and that of 30 kb was approximately 5%. In the control strain PB-cas12a, the knockout efficiency of 9-16 kb large-fragment genes was approximately 60-80%, that of 20 kb was approximately 5%, and that of 25 kb and 30 kb was almost 0.

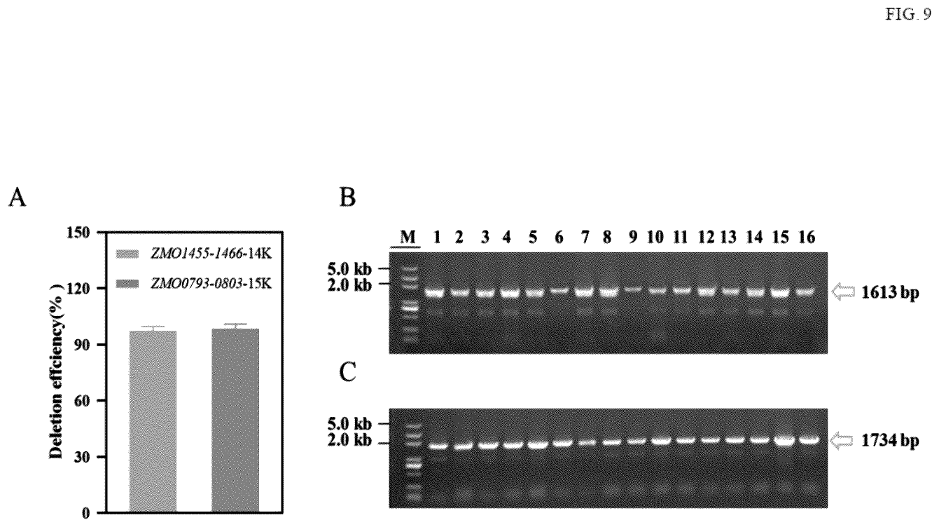

PB-cas12a_Pt-recET was further used to knock out the transporter/secretion protein gene clusters ZMO1455-1466 (14 kb) and ZMO0793-0803 (15 kb), and colony PCR was used to verify the knockout efficiency. The results are shown in FIG. 9, where FIG. A shows the knockout efficiency of the ZMO1455-1466 and ZMO0793-0803 gene clusters, and FIGS. B—C show the colony PCR amplification results of the ZMO1455-1466 and ZMO0793-0803 gene clusters, respectively. The results showed that the knockout efficiency of both gene clusters exceeded 95%.

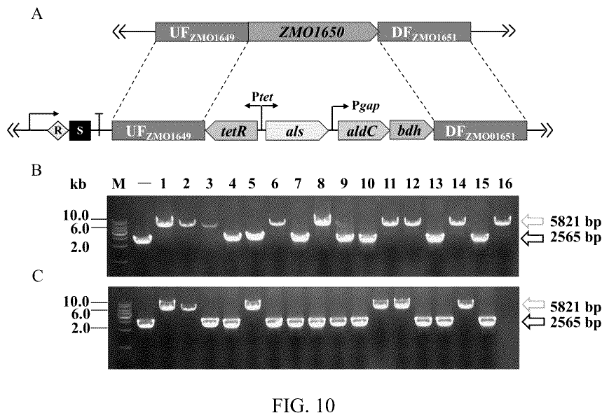

In order to test the knock-in efficiency of large fragments in PB-cas12a_Pt-recET, the 2,3-butanediol metabolic pathway Ptet-als Pgap-aldC_bdh was selected for knock-in at the genomic ZMO1650 locus (see FIG. 10A). The editing plasmid pEZTH-2,3-BDO-1650 targeting ZMO1650 was constructed and transformed into PB-cas12a_Pt-recET and the control strain PB-cas12a. Colony PCR was used to verify the knock-in efficiency, and the results are shown in FIGS. 10A and 10B. The results showed that the knock-in efficiency of the 2,3-butanediol pathway gene cluster in PB-cas12a_Pt-recET was approximately 56.25%, and that of the 2,3-butanediol pathway gene cluster in the control strain was approximately 37.50%, indicating that the application of the CRISPR-Cas12a combined with the RecET system is beneficial to improving the insertion efficiency of large-fragment genes in the genome.

5. Method for Eliminating the Editing Plasmid in Zymomonas mobilis PB-Cas12a_Pt-recET

The PB-cas12a_Pt-recET strain was inoculated into RMG5 liquid medium without resistance. After growing to turbidity, 100 μL of the bacterial solution was transferred to 1 mL of fresh RMG5 liquid medium. After 4-5 generations of continuous subculture, 100 μL of the bacterial solution was diluted and spread on RMG5 plates. After single colonies grew on the plate, colony PCR was performed on the single colonies using primers for verifying the editing plasmid. If no PCR band was observed, the editing plasmid might have been lost. The single colonies without PCR bands were inoculated into RMG5 liquid medium and RMG5+Cm liquid medium, respectively, and cultured statically at 30° C. The next day, the culture results in the two media were observed. If the strain grew turbid in RMG5 liquid medium, but could not grow and remained clear in RMG5+Cm liquid medium, it could be confirmed that the editing plasmid had been eliminated.

The foregoing description merely describes the embodiments of the present disclosure and is not intended to limit the present disclosure. Any modifications, equivalent substitutions and improvements made within the spirit and principles of the present disclosure shall fall within the scope of protection of the present disclosure.

What is claimed is:

1. A method for constructing a Zymomonas mobilis chassis cell, wherein the chassis cell uses Zymomonas mobilis ZMNP-Cas12a with the Cas12a gene integrated at the genomic ZMO0038 locus as a starting strain, integrates the RecET gene into the ZMO0028 locus of the genome of Zymomonas mobilis ZMNP-Cas12a, and the upstream of the RecET gene is linked to a Pₜₑₜ promoter, and the upstream of the Cas12a gene is linked to a P_{BAD} promoter; wherein, the nucleotide sequence of the Cas12a gene is shown in SEQ ID NO.1, the nucleotide sequence of the RecET gene is shown in SEQ ID NO.2, the nucleotide sequence of the P_{d} promoter is shown in SEQ ID NO.3, and the nucleotide sequence of the P_{BAD} promoter is shown in SEQ ID NO.4;
the method for constructing the chassis cell comprises the following steps:
obtaining the upstream homologous arm 0038US and downstream homologous arm 0038DS sequences of the genomic ZMO0038 locus, tandemly linking the P_{BAD} promoter and Cas12a gene between the 0038US and 0038DS sequences, and constructing a first homologous recombination plasmid;
obtaining the upstream homologous arm 0028US and downstream homologous arm 0028DS sequences of the genomic ZMO0028 locus, tandemly linking the Pₜₑₜ promoter and RecET gene between the 0028US and 0028DS sequences, and constructing a second homologous recombination plasmid; and
transforming the first homologous recombination plasmid and the second homologous recombination plasmid into Zymomonas mobilis ZMNP-Cas12a respectively, integrating the P_{BAD} promoter and the Cas12a gene into the ZMO0038 locus and integrating the Pₜₑₜ promoter and the RecET gene into the ZMO0028 locus through homologous recombination, and screening to obtain the Zymomonas mobilis chassis cell.

2. The method for constructing the Zymomonas mobilis chassis cell according to claim 1, wherein the first homologous recombination plasmid and the second homologous recombination plasmid carry different resistance marker genes.

3. The method for constructing the Zymomonas mobilis chassis cell according to claim 1, wherein the first homologous recombination plasmid and the second homologous recombination plasmid further comprise an Ori sequence derived from a pUC57 vector.

4. The method for constructing the Zymomonas mobilis chassis cell according to claim 3, wherein the construction of the first homologous recombination plasmid comprises the following steps: obtaining the upstream homologous arm 0038US and downstream homologous arm 0038DS sequences of the ZMO0038 locus by PCR amplification, and obtaining the P_{BAD} promoter, Cas12a gene, spectinomycin gene, and Ori sequence of the pUC57 vector by PCR amplification, and connecting the amplified 0038US sequence, Cas12a gene, P_{BAD} promoter, spectinomycin gene, and 0038DS sequence with the Ori sequence through Gibson assembly to obtain the first homologous recombination plasmid;
the construction of the second homologous recombination plasmid comprises the following steps: obtaining the upstream homologous arm 0028US and downstream homologous arm 0028DS sequences of the ZMO0028 locus by PCR amplification, and obtaining the Pt promoter, RecET gene, chloramphenicol gene, and Ori sequence of the pUC57 vector by PCR amplification, and connecting the amplified 0028US sequence, RecET gene, Pt promoter, chloramphenicol gene, and 0028DS sequence with the Ori sequence through Gibson assembly to obtain the second homologous recombination plasmid;
wherein, the nucleotide sequences of the primer pairs for amplifying the 0038US sequence are shown in SEQ ID NO.20-21, the nucleotide sequences of the primer pairs for amplifying the 0038DS sequence are shown in SEQ ID NO.22-23, the nucleotide sequences of the primer pairs for amplifying the 0028US sequence are shown in SEQ ID NO.10-11, the nucleotide sequences of the primer pairs for amplifying the 0028DS sequence are shown in SEQ ID NO.12-13, the nucleotide sequences of the primer pairs for amplifying the Cas12a gene are shown in SEQ ID NO.18-19, the nucleotide sequences of the primer pairs for amplifying the RecET gene are shown in SEQ ID NO.5-6, the nucleotide sequences of the primer pairs for amplifying the P_{BAD} promoter are shown in SEQ ID NO.7 and 9, the nucleotide sequences of the primer pairs for amplifying the Pt promoter are shown in SEQ ID NO.7-8, the nucleotide sequences of the primer pairs for amplifying the spectinomycin gene are shown in SEQ ID NO.24-25, the nucleotide sequences of the primer pairs for amplifying the chloramphenicol gene are shown in SEQ ID NO.14-15, and the nucleotide sequences of the primer pairs for amplifying the Ori sequence are shown in SEQ ID NO.16-17.

5. Use of the Zymomonas mobilis chassis cell constructed by the method for constructing the Zymomonas mobilis chassis cell according to claim 1 in large-fragment gene editing, wherein the gene sequence length ranges from 9 to 25 kb.

6. The use of the Zymomonas mobilis chassis cell according to claim 5 in large-fragment gene editing, wherein the gene editing is gene knockout, gene replacement, and/or point mutation.

7. A gene editing method for a Zymomonas mobilis chassis cell, comprising the following steps:
designing guide RNA and donor DNA according to the target sequence to be edited, wherein the donor DNA comprises upstream homologous arm and downstream homologous arm sequences of the guide RNA targeting sequence, linking the guide RNA and donor DNA to an expression vector to obtain an editing plasmid; and
transforming the editing plasmid into the Zymomonas mobilis chassis cell constructed by the method for constructing the Zymomonas mobilis chassis cell of claim 1, and culturing positive chassis cells transformed with the editing plasmid.
